(19) United States
(12) Patent Application Publication
(10) Pub. No.: US 2014/0024653 A1
(43) Pub. Date: Jan. 23, 2014

(54) COMPOSITIONS AND METHODS FOR TREATING CANCER USING PI3K INHIBITOR AND MEK INHIBITOR

(71) Applicants: Merck Patent GmbH, Darmstadt (DE); Sanofi, Paris (FR)

(72) Inventors: Laurent DEBUSSCHE, Athis Mons (FR); Carlos GARCIA-ECHEVERRIA, St. Cloud (FR); Jianguo MA, Newton, PA (US); Stuart McMillan, Cambridge, MA (US); Janet Ann Meurer OGDEN, Westwood, MA (US); Loic VINCENT, Vitry-Sur-Seine (FR)

(73) Assignees: Merck Patent GmbH, Darmstadt (DE); Sanofi, Paris (FR)

(21) Appl. No.: 13/912,647

(22) Filed: Jun. 7, 2013

Related U.S. Application Data
(63) Continuation of application No. PCT/US2011/063871, filed on Dec. 8, 2011.

(30) Foreign Application Priority Data
Nov. 3, 2011 (FR) 1159940

(51) Int. Cl. A61K 31/4985; A61K 31/44; A61K 31/498

(52) U.S. Cl. CPC A61K 31/4985; A61K 31/498; A61K 31/44
USPC 514/249

(57) ABSTRACT

Methods of treating patients with cancer are provided, wherein the methods comprise administering to the patient an effective amount of a MEK inhibitor and an effective amount of a PI3K inhibitor. Compositions in which the MEK and PI3K inhibitors are combined also are described.

### CROSS-REFERENCE TO RELATED APPLICATIONS

This application is a Continuation of International Application No. PCT/US11/63781, filed Dec. 8, 2011, which claims the benefit of priority of U.S. Provisional Application No. 61/421,465 filed Dec. 9, 2010, U.S. Provisional Application No. 61/436,258 filed Jan. 26, 2011, and U.S. Provisional Application No. 61/467,485 filed Mar. 25, 2011, all of which are incorporated herein by reference in their entireties.

### BACKGROUND

There is an ongoing need in the art for more efficacious methods and compositions in the treatment of cancer. The instant application is directed, generally, to compositions and methods for the treatment of cancer, and more particularly, to compositions and methods comprising inhibitors of the mitogen activated protein kinase (MEK) and/or phosphoinositide 3-kinase (PI3K) pathways.

Tumor cells treated with inhibitors of MEK kinases typically respond via inhibition of phosphorylation of ERK, down-regulation of Cyclin D, induction of G1 arrest, and finally undergoing apoptosis. Pharmacologically, MEK inhibition completely abrogates tumor growth in BRaf xenograft tumors whereas Ras mutant tumors exhibit only partial inhibition in most cases (D. B. Solit et al., Nature 2006; 439: 358-362). Thus, MEKs have been targets of great interest for the development of cancer therapeutics.

N—((S)-2,3-dihydroxypropyl)-3-(2-fluoro-4-iodo-phenylamino)isonicotinamide (also referred to as MSC1936369 or AS703026) is a novel, allosteric inhibitor of MEK. It possesses relatively high potency and selectivity, having no activity against 217 kinases or 90 non-kinase targets when tested at 10 μM. The in vivo PK profile of AS703026 is acceptable in mice and rats, with relatively high oral bioavailability (52-57%), medium or high clearance (0.9-2.6 L/h/kg) and medium or long half-life (2.2-4.7 h). The compound is relatively well-tolerated in mice, with a two-week maximum tolerated dose of 60 mg/kg BID.

N-(3-{[(3-{[2-chloro-5-(methoxy)phenyl]amino}quinoxalin-2-yl)amino]sulfonyl}phenyl)-2-methylalaninamide (also known as XL147 or SAR245408) and 2-amino-8-ethyl-4-methyl-6-(1H-pyrazol-5-yl)pyrido[2,3-d]pyrimidin-7(8H)-one (also known as XL765 or SAR245409) are selective inhibitors of class I PI3K lipid kinases. XL147 inhibits the phosphorylation of downstream effectors Akt and S6 ribosomal protein (S6RP) and targets only PI3K isoforms (inhibitor concentration, i.e., IC₅₀ values in nanomolar (nM): PI3Kα 39, PI3Kβ 383, PI3Kδ 36, PI3Kγ 23). XL765 targets both PI3K isoforms (IC₅₀ values in nM: PI3Kα 39, PI3Kβ 113, PI3Kδ 43, PI3Kγ 9) and mTOR (157 nM).

Oral administration of XL147 or XL765 alone inhibits tumor growth in mice bearing xenografts in which PI3K signaling is activated, such as the PTEN-deficient PC-3 prostate adenocarcinoma, U87-MG gliobastoma, A2058 melanoma and WM-266-4 melanoma, or the PIK3CA mutated MCF7 mammary carcinoma. XL147 is currently undergoing several Phase I trials for patients with solid tumors and/or lymphoma and Phase II trials for patients with endometrial or hormone receptor-positive breast cancer. XL765 is currently undergoing testing in Phase I clinical trials for patients with solid tumor, lymphoma or glioblastoma and in a Phase I/II trial for patients with hormone receptor-positive breast cancer.

There remains a need, however, for a cancer therapy that is more effective in inhibiting cell proliferation and tumor growth while minimizing patient toxicity. There is a particular need for an MEK or PI3K inhibitor therapy is made more efficacious without substantially increasing, or even maintaining or decreasing, the dosages of MEK or PI3K inhibitor traditionally employed in the art.

### SUMMARY

In one aspect, there is provided compositions and uses thereof in the treatment of a variety of cancers.

In particular embodiments, there is provided a composition that includes a compound having the following structural formula:

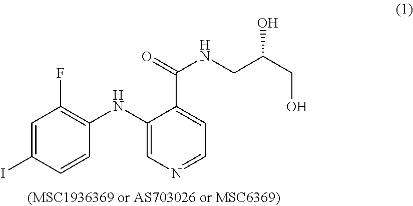 [SMILES: O=C(NC[C@H](O)CO)c1ccncc1Nc1ccc(I)cc1F]

and a compound selected from the group consisting of

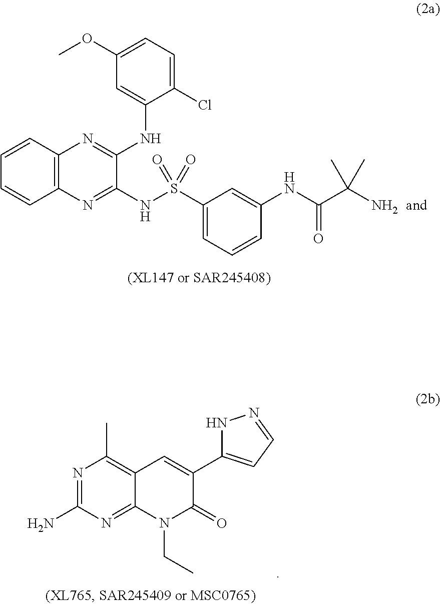 [SMILES: COc1ccc(Cl)c(Nc2nc3ccccc3nc2NS(=O)(=O)c2cccc(NC(=O)C(C)(C)N)c2)c1 ; CCn1c(=O)c(-c2ccn[nH]2)cc2c(C)nc(N)nc21]

In another aspect, methods of treating a patient with cancer are provided that comprise administering to the patient a therapeutically effective amount of a compound of Formula (1), or a pharmaceutically acceptable salt thereof, in combination with the compound of Formula (2a) or Formula (2b), or a pharmaceutically acceptable salt thereof.

In one embodiment, a method of treating a patient with cancer comprises administering to the patient a first dosage of a MEK inhibitor and a second dosage of a PI3K inhibitor, wherein said MEK inhibitor has the following structural formula:

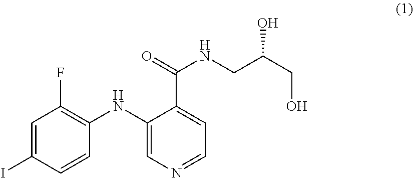 [SMILES: O=C(NC[C@H](O)CO)c1ccncc1Nc1ccc(I)cc1F]

and said PI3K inhibitor is selected from the group consisting of

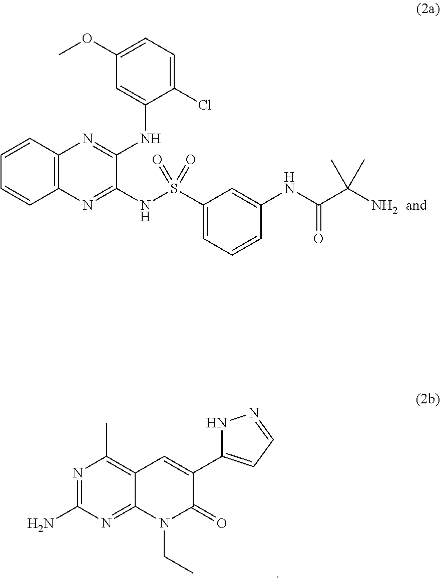 [SMILES: COc1ccc(Cl)c(Nc2nc3ccccc3nc2NS(=O)(=O)c2cccc(NC(=O)C(C)(C)N)c2)c1 ; CCn1c(=O)c(-c2ccn[nH]2)cc2c(C)nc(N)nc21]

In some embodiments, the methods involve treating cancer selected from the group consisting of non-small cell lung cancer, breast cancer, pancreatic cancer, liver cancer, prostate cancer, bladder cancer, cervical cancer, thyroid cancer, colorectal cancer, liver cancer, muscle cancer, hematological malignancies, melanoma, endometrial cancer and pancreatic cancer. In others, the cancer is selected from the group consisting of colorectal cancer, endometrial cancer, hematological malignancies, thryoid cancer, breast cancer, melanoma, pancreatic cancer and prostate cancer.

In some embodiments, the compositions and methods of use described herein are in amounts (i.e., either in the composition are in an administered dosage) that synergistically reduce tumor volume in a patient. In further embodiments, the synergistic combination achieves tumor stasis or tumor regression.

In another aspect, a combination for use in treating cancer is provided, the combination comprising a therapeutically effective amount of (A) the compound of Formula (1), or a pharmaceutically acceptable salt thereof, and (B) the compound of Formula (2a) or Formula (2b), or a pharmaceutically acceptable salt thereof.

In one embodiment, uses of a combination comprising a therapeutically effective amount of (A) the compound of Formula (1), or a pharmaceutically acceptable salt thereof, and (B) the compound of Formula (2a) or Formula (2b), or a pharmaceutically acceptable salt thereof, are provided for the preparation of a medicament for use in treatment of cancer.

In another aspect, kits are provided comprising: (A) the compound of Formula (1), or a pharmaceutically acceptable salt thereof; (B) the compound of Formula (2a) or Formula (2b), or a pharmaceutically acceptable salt thereof; and (C) instructions for use.

Other objects, features and advantages will become apparent from the following detailed description. The detailed description and specific examples are given for illustration only since various changes and modifications within the spirit and scope of the invention will become apparent to those skilled in the art from this detailed description. Further, the examples demonstrate the principle of the invention and cannot be expected to specifically illustrate the application of this invention to all the examples where it will be obviously useful to those skilled in the prior art.

### BRIEF DESCRIPTION OF THE DRAWINGS

FIG. 1 provides a plot showing body weight change during the evaluation of the antitumor activity of Compound (1) (5 mg/kg) in combination with Compound (2b) (30 mg/kg) and Compound (2a) (50 and 75 mg/kg) against human HCT 116 bearing SCID female mice.

FIG. 2 provides a plot showing antitumor activity of Compound (1) (5 mg/kg) in combination with Compound (2b) (30 mg/kg) against human HCT 116 bearing SCID female mice.

FIG. 3 provides a plot showing antitumor activity of Compound (1) (5 mg/kg) in combination with Compound (2a) (50 and 75 mg/kg) against human HCT 116 bearing SCID female mice. The box indicates combinations achieving therapeutic synergy.

FIG. 4 provides a plot showing body weight change during the evaluation of the antitumor activity of Compound (1) (10 and 20 mg/kg) in combination with Compound (2b) (20 mg/kg) and Compound (2a) (50 and 75 mg/kg) against human HCT 116 bearing SCID female mice.

FIG. 5 provides a plot showing antitumor activity of Compound (1) (10 and 20 mg/kg) in combination with Compound (2b) (20 mg/kg) against human HCT 116 bearing SCID female mice.

FIG. 6 provides a plot showing antitumor activity of Compound (1) (10 mg/kg) in combination with Compound (2a) (50 and 75 mg/kg) against human HCT 116 bearing SCID female mice.

FIG. 7 provides a plot showing body weight change during the evaluation of the antitumor activity of Compound (1) (10 and 20 mg/kg) in combination with Compound (2a) (50 and 75 mg/kg) against human HCT 116 bearing SCID female mice.

FIG. 8 provides a plot showing antitumor activity of Compound (1) (10 and 20 mg/kg) in combination with Compound (2a) (50 and 75 mg/kg) against human HCT 116 bearing SCID female mice. The box indicates combinations achieving therapeutic synergy.

FIG. 9 provides a plot showing body weight change during the evaluation of the antitumor activity of Compound (1) (10 and 20 mg/kg) in combination with Compound (2b) (20 mg/kg) against human HCT 116 bearing SCID female mice.

FIG. 10 provides a plot showing antitumor activity of Compound (1) (10 and 20 mg/kg) in combination with Compound (2b) (20 mg/kg) against human HCT 116 bearing SCID female mice.

FIG. 11 provides a plot showing percent body weight of MiaPaCa-2 tumor-bearing mice treated with Compound (1) (5 mg/kg) and Compound (2a) (50 mg/kg) alone or in combination.

FIG. 12 provides a plot showing percent body weight of MiaPaCa-2 tumor-bearing mice treated with Compound (1) (5 mg/kg) and Compound (2b) (30 mg/kg) alone or in combination.

FIG. 13 provides a plot showing mean tumor volumes of MiaPaCa-2 tumor-bearing mice treated with Compound (1) (5 mg/kg) and Compound (2a) (50 mg/kg) alone or in combination.

FIG. 14 provides a plot showing mean tumor volumes of MiaPaCa-2 tumor-bearing mice treated with Compound (1) (5 mg/kg) and Compound (2b) (30 mg/kg) alone or in combination.

FIGS. 15A, 15B-1 and 15B-2 provide charts showing Z-score values of Compound (1) for various tumor cell lines identifying specific therapeutic applications. Selection of specific therapeutic applications for Compound (1). Individual z-score values for each cell line are plotted within one group corresponding to the tumor origin. An average value for all values within one group is shown as a triangle, and can serve as an indicator for Compound (1) activity within one group. As for individual z-scores, z-scores below mean strong efficacy, whereas z-scores >0 approximate resistance.

FIGS. 16A, 16B-1 and 16B-2 provide charts showing Z-score values of Compound (2b) for various tumor cell lines identifying specific therapeutic applications. Selection of specific therapeutic applications for Compound (2b). Individual z-score values for each cell line are plotted within one group corresponding to the tumor origin. An average value for all values within one group is shown as a triangle and can serve as an indicator for Compound (2b) activity within one group. As for individual z-scores, z-scores below zero mean strong efficacy, whereas a z-score >0 approximate resistance.

FIGS. 17-A and 17-B provide a chart showing Z-score values of Compound (1) in combination with Compound (2b) for various tumor cell lines.

FIGS. 18A, 18B, 18C, 18D, 18E and 18F provide plots and graphs showing combination results of Compound (1) with Compound (2b) in CRC tumor cell lines (synergy plot & mutation analysis).

FIGS. 19A and 19B provide plots and graphs showing combination results of Compound (1) with Compound (2b) in pancreatic tumor cell lines (synergy plot & mutation analysis).

FIGS. 20A and 20B provide plots and graphs showing combination results of Compound (1) with Compound (2b) in NSCLC tumor cell lines (synergy plot & mutation analysis).

FIG. 21 provides a plot showing body weight change during the evaluation of the antitumor activity of Compound (1) (20 mg/kg) in combination with Compound (2b) (20 mg/kg) and Compound (2a) (75 mg/kg) against human primary colon tumors CR-LRB-009C bearing SCID female mice.

FIG. 22 provides a plot showing antitumor activity of Compound (1) (20 mg/kg) in combination with Compound (2b) (20 mg/kg) and Compound (2a) (75 mg/kg) against human primary colon tumors CR-LRB-009C bearing SCID female mice.

FIG. 23 provides a plot showing body weight change during the evaluation of the antitumor activity of Compound (1) (20 mg/kg) in combination with Compound (2b) (20 mg/kg) and Compound (2a) (75 mg/kg) against human primary colon tumors CR-LRB-013P bearing SCID female mice.

FIG. 24 provides a plot showing antitumor activity of Compound (1) (20 mg/kg) in combination with Compound (2b) (20 mg/kg) and Compound (2a) (75 mg/kg) against human primary colon tumors CR-LRB-013P bearing SCID female mice.

FIG. 25 graphically depicts the results of Icyte ex vivo imaging of Evans Blue tumor extravasation performed after treatment with either Compound (2a) or Compound (2b) as single agents or in combination with Compound (1) in HCT116 xenografts.

FIGS. 26A and 26B graphically depict results of FMT imaging after three days of therapy, three hours after AnnexinV-750 administration, four hours post-treatment with Compound (1), Compound (2a) or Compound (2b) as single agents or combinations in HCT116 xenografts. Tumor fluorescence was quantified in pmol of fluorophore and standardized to the tumor volume. Statistics: Newman-Keuls after 2way Anova on Ranked data, NS: P<0.05).

FIGS. 27A and 27B graphically show protein levels of cleaved-PARP and caspase-3 in tumor extracts following treatment with Compound (1), Compound (2a) or Compound (2b) alone or in selected combination. Statistics: Dunnett's test for one factor after one way Anova, NS: P<0.05.

FIG. 28 provides a plot showing tumor volumes of HCT116 tumor-bearing mice treated with Compound (1) (10 mg/kg), Compound (2a) (50 mg/kg) or Compound (2b)(20 mg/kg) alone or in combination. To quantify apoptosis, fluorescent Annexin-Vivo-750 was injected iv on day 3 and day 7 after start of treatment, 1 hour post daily treatment. Animals were imaged by FMT 3 hours post probe injection.

### DETAILED DESCRIPTION

In one aspect, methods for treating patients with cancer are provided. In one embodiment, the methods comprise administering to the patient a therapeutically effective amount of a MEK inhibitor and a therapeutically effective amount of a PI3K inhibitor, as further described below.

In one embodiment, the inventive methods and compositions comprise a MEK inhibitor having the following structural formula:

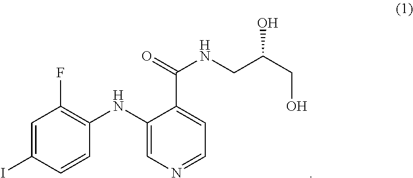 [SMILES: O=C(NC[C@H](O)CO)c1ccncc1Nc1ccc(I)cc1F]

The MEK inhibitor according to formula (1), is referred to herein as “Compound (1)” and is known also as MSC1936369, AS703026 or MSC6369. The preparation, properties, and MEK-inhibiting abilities of Compound (1) are provided in, for example, International Patent Publication No. WO 06/045514, particularly Example 115 and Table 1 therein. The entire contents of WO 06/045514 are incorporated herein by reference. Neutral and salt forms of the compound of Formula (1) are all considered herein.

In other embodiments, the inventive methods and compositions comprise a PI3K inhibitor having one of the following structures:

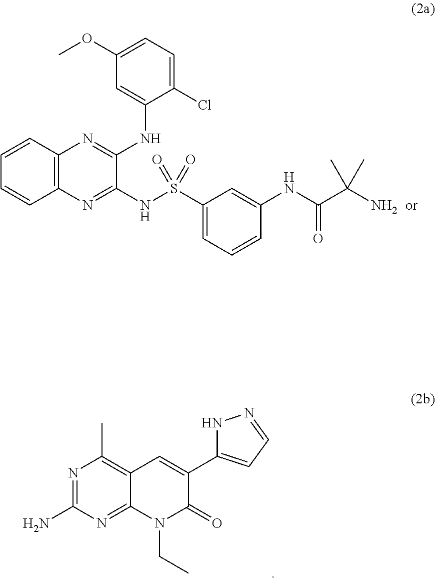 [SMILES: COc1ccc(Cl)c(Nc2nc3ccccc3nc2NS(=O)(=O)c2cccc(NC(=O)C(C)(C)N)c2)c1 ; CCn1c(=O)c(-c2ccn[nH]2)cc2c(C)nc(N)nc21]

The PI3K inhibitor according to formula (2a), is referred to herein as “Compound (2a)” and is known also as XL147 or SAR245408. The PI3K inhibitor according to formula (2b), is referred to herein as “Compound (2b)” and is known also as XL765, SAR245409 or MSC0765. The preparation and properties of Compound (2a) are provided in, for example, International Patent Publication No. WO 07/044,729, particularly Example 357 therein. The entire contents of WO 07/044,729 are incorporated herein by reference. The preparation and properties of Compound (2b) are provided in, for example, International Patent Publication No. WO 07/044,813, particularly Example 56 therein. The entire contents of WO 07/044,813 are incorporated herein by reference.

In some embodiments, the compounds described above are unsolvated. In other embodiments, one or both of the compounds used in the method are in solvated form. As known in the art, the solvate can be any of pharmaceutically acceptable solvent, such as water, ethanol, and the like. In general, the presence of a solvate or lack thereof does not have a substantial effect on the efficacy of the MEK or PI3K inhibitor described above.

Although the compounds in Formula (1), Formula (2a) and Formula (2b) are depicted in their neutral forms, in some embodiments, these compounds are used in a pharmaceutically acceptable salt form. The salt can be obtained by any of the methods well known in the art, such as any of the methods and salt forms elaborated upon in WO 07/044,729, as incorporated by reference herein. A “pharmaceutically acceptable salt” of the compound refers to a salt that is pharmaceutically acceptable and that retains pharmacological activity. It is understood that the pharmaceutically acceptable salts are non-toxic. Additional information on suitable pharmaceutically acceptable salts can be found in Remington's Pharmaceutical Sciences, 17th ed., Mack Publishing Company, Easton, Pa., 1985, or S. M. Berge, et al., “Pharmaceutical Salts,” J. Pharm. Sci., 1977; 66:1-19, both of which are incorporated herein by reference.

Examples of pharmaceutically acceptable acid addition salts include those formed with inorganic acids such as hydrochloric acid, hydrobromic acid, sulfuric acid, nitric acid, phosphoric acid, as well as those salts formed with organic acids, such as acetic acid, trifluoroacetic acid, propionic acid, hexanoic acid, cyclopentanepropionic acid, glycolic acid, pyruvic acid, lactic acid, oxalic acid, maleic acid, malonic acid, succinic acid, fumaric acid, tartaric acid, citric acid, benzoic acid, cinnamic acid, 3-(4-hydroxybenzoyl)benzoic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, p-toluenesulfonic acid, and salicylic acid.

In a first set of embodiments, the MEK inhibitor of formula (1) is administered simultaneously with the PI3K inhibitor of either formula (2a) or (2b). Simultaneous administration typically means that both compounds enter the patient at precisely the same time. However, simultaneous administration also includes the possibility that the MEK inhibitor and PI3K inhibitor enter the patient at different times, but the difference in time is sufficiently miniscule that the first administered compound is not provided the time to take effect on the patient before entry of the second administered compound. Such delayed times typically correspond to less than 1 minute, and more typically, less than 30 seconds.

In one example, wherein the compounds are in solution, simultaneous administration can be achieved by administering a solution containing the combination of compounds. In another example, simultaneous administration of separate solutions, one of which contains the MEK inhibitor and the other of which contains the PI3K inhibitor, can be employed. In one example wherein the compounds are in solid form, simultaneous administration can be achieved by administering a composition containing the combination of compounds.

In other embodiments, the MEK and PI3K inhibitors are not simultaneously administered. In this regard, the first administered compound is provided time to take effect on the patient before the second administered compound is administered. Generally, the difference in time does not extend beyond the time for the first administered compound to complete its effect in the patient, or beyond the time the first administered compound is completely or substantially eliminated or deactivated in the patient. In one set of embodiments, the MEK inhibitor is administered before the PI3K inhibitor. In another set of embodiments, the PI3K inhibitor is administered before the MEK inhibitor. The time difference in non-simultaneous administrations is typically greater than 1 minute, and can be, for example, precisely, at least, up to, or less than 5 minutes, 10 minutes, 15 minutes, 30 minutes, 45 minutes, 60 minutes, two hours, three hours, six hours, nine hours, 12 hours, 24 hours, 36 hours, or 48 hours.

In one set of embodiments, one or both of the MEK and PI3K inhibitors are administered in a therapeutically effective (i.e., therapeutic) amount or dosage. A “therapeutically effective amount” is an amount of the MEK or PI3K inhibitor that, when administered to a patient by itself, effectively treats the cancer (for example, inhibits tumor growth, stops tumor growth, or causes tumor regression). An amount that proves “therapeutically effective amount” in a given instance, for a particular subject, may not be effective for 100% of subjects similarly treated for the disease or condition under consideration, even though such dosage is deemed a “therapeutically effective amount” by skilled practitioners. The amount of the compound that corresponds to a therapeutically effective amount is strongly dependent on the type of cancer, stage of the cancer, the age of the patient being treated, and other facts. In general, therapeutically effective amounts of these compounds are well-known in the art, such as provided in the supporting references cited above.

In another set of embodiments, one or both of the MEK and PI3K inhibitors are administered in a sub-therapeutically effective amount or dosage. A sub-therapeutically effective amount is an amount of the MEK or PI3K inhibitor that, when administered to a patient by itself, does not completely inhibit over time the biological activity of the intended target.

Whether administered in therapeutic or sub-therapeutic amounts, the combination of MEK inhibitor and PI3K inhibitor should be effective in treating the cancer. A sub-therapeutic amount of MEK inhibitor can be an effective amount if, when combined with the PI3K inhibitor, the combination is effective in the treatment of a cancer.

In some embodiments, the combination of compounds exhibits a synergistic effect (i.e., greater than additive effect) in treating the cancer, particularly in reducing a tumor volume in the patient. In different embodiments, depending on the combination and the effective amounts used, the combination of compounds can either inhibit tumor growth, achieve tumor stasis, or even achieve substantial or complete tumor regression.

In some embodiments, Compound (1) is administered at a dosage of about 7-120 mg po qd. Compound (2a), meanwhile, can be administered at a dosage of about 12-600 mg po qd. Compound (2b) can be administered at a dosage of about 15-90 mg po qd.

As used herein, the term “about” generally indicates a possible variation of no more than 10%, 5%, or 1% of a value. For example, “about 25 mg/kg” will generally indicate, in its broadest sense, a value of 22.5-27.5 mg/kg, i.e., 25±10 mg/kg.

While the amounts of MEK and PI3K inhibitors should result in the effective treatment of a cancer, the amounts, when combined, are preferably not excessively toxic to the patient (i.e., the amounts are preferably within toxicity limits as established by medical guidelines). In some embodiments, either to prevent excessive toxicity and/or provide a more efficacious treatment of the cancer, a limitation on the total administered dosage is provided. Typically, the amounts considered herein are per day; however, half-day and two-day or three-day cycles also are considered herein.

Different dosage regimens may be used to treat the cancer. In some embodiments, a daily dosage, such as any of the exemplary dosages described above, is administered once, twice, three times, or four times a day for three, four, five, six, seven, eight, nine, or ten days. Depending on the stage and severity of the cancer, a shorter treatment time (e.g., up to five days) may be employed along with a high dosage, or a longer treatment time (e.g., ten or more days, or weeks, or a month, or longer) may be employed along with a low dosage. In some embodiments, a once- or twice-daily dosage is administered every other day. In some embodiments, each dosage contains both the MEK and PI3K inhibitors, while in other embodiments, each dosage contains either the MEK or PI3K inhibitors. In yet other embodiments, some of the dosages contain both the MEK and PI3K inhibitors, while other dosages contain only the MEK or the PI3K inhibitor.

Examples of types of cancers to be treated with the present invention include, but are not limited to, lymphomas, sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, synovioma, mesothelioma, lymphangioendotheliosarcoma, Ewing's tumor, leiomyosarcoma, rhabdomyosarcoma, colon carcinoma, pancreatic cancer, breast cancer, ovarian cancer, prostate cancer, gastric cancer, esophageal cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, non-small cell lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias, e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrom's macroglobulinemia and heavy chain disease.

In some embodiments, the cancer being treated is selected from the group consisting of non-small cell lung cancer, breast cancer, pancreatic cancer, liver cancer, prostate cancer, bladder cancer, cervical cancer, thyroid cancer, colorectal cancer, liver cancer, and muscle cancer. In other embodiments, the cancer is selected from colorectal cancer, endometrial cancer, hematology cancer, thryoid cancer, triple negative breast cancer or melanoma.

The patient considered herein is typically a human. However, the patient can be any mammal for which cancer treatment is desired. Thus, the methods described herein can be applied to both human and veterinary applications.

The term “treating” or “treatment”, as used herein, indicates that the method has, at the least, mitigated abnormal cellular proliferation. For example, the method can reduce the rate of tumor growth in a patient, or prevent the continued growth of a tumor, or even reduce the size of a tumor.

In another aspect, methods for preventing cancer in an animal are provided. In this regard, prevention denotes causing the clinical symptoms of the disease not to develop in an animal that may be exposed to or predisposed to the disease but does not yet experience or display symptoms of the disease. The methods comprise administering to the patient a MEK inhibitor and a PI3K inhibitor, as described herein. In one example, a method of preventing cancer in an animal comprises administering to the animal a compound of Formula (1), or a pharmaceutically acceptable salt thereof, in combination with a compound selected from the group consisting of Formula (2a) and Formula (2b), or a pharmaceutically acceptable salt thereof.

The MEK and PI3K inhibiting compounds, or their pharmaceutically acceptable salts or solvate forms, in pure form or in an appropriate pharmaceutical composition, can be administered via any of the accepted modes of administration or agents known in the art. The compounds can be administered, for example, orally, nasally, parenterally (intravenous, intramuscular, or subcutaneous), topically, transdermally, intravaginally, intravesically, intracistemally, or rectally. The dosage form can be, for example, a solid, semi-solid, lyophilized powder, or liquid dosage forms, such as for example, tablets, pills, soft elastic or hard gelatin capsules, powders, solutions, suspensions, suppositories, aerosols, or the like, preferably in unit dosage forms suitable for simple administration of precise dosages. A particular route of administration is oral, particularly one in which a convenient daily dosage regimen can be adjusted according to the degree of severity of the disease to be treated.

In another aspect, the instant application is directed to a composition that includes the MEK inhibitor shown in Formula (1) and a PI3K inhibitor selected from the compounds shown in Formulas (2a) and (2b). In some embodiments, the composition includes only the MEK and PI3K inhibitors described above. In other embodiments, the composition is in the form of a solid (e.g., a powder or tablet) including the MEK and PI3K inhibitors in solid form, and optionally, one or more auxiliary (e.g., adjuvant) or pharmaceutically active compounds in solid form. In other embodiments, the composition further includes any one or combination of pharmaceutically acceptable carriers (i.e., vehicles or excipients) known in the art, thereby providing a liquid dosage form.

Auxiliary and adjuvant agents may include, for example, preserving, wetting, suspending, sweetening, flavoring, perfuming, emulsifying, and dispensing agents. Prevention of the action of microorganisms is generally provided by various antibacterial and antifungal agents, such as, parabens, chlorobutanol, phenol, sorbic acid, and the like. Isotonic agents, such as sugars, sodium chloride, and the like, may also be included. Prolonged absorption of an injectable pharmaceutical form can be brought about by the use of agents delaying absorption, for example, aluminum monostearate and gelatin. The auxiliary agents also can include wetting agents, emulsifying agents, pH buffering agents, and antioxidants, such as, for example, citric acid, sorbitan monolaurate, triethanolamine oleate, butylated hydroxytoluene, and the like.

Dosage forms suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, cellulose derivatives, starch, alignates, gelatin, polyvinylpyrrolidone, sucrose, and gum acacia, (c) humectants, as for example, glycerol, (d) disintegrating agents, as for example, agar-agar, calcium carbonate, potato or tapioca starch, alginic acid, croscarmellose sodium, complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, magnesium stearate and the like (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms also may comprise buffering agents.

Solid dosage forms as described above can be prepared with coatings and shells, such as enteric coatings and others well-known in the art. They can contain pacifying agents and can be of such composition that they release the active compound or compounds in a certain part of the intestinal tract in a delayed manner. Examples of embedded compositions that can be used are polymeric substances and waxes. The active compounds also can be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, dispersing, etc., a MEK or PI3K inhibitor compound described herein, or a pharmaceutically acceptable salt thereof, and optional pharmaceutical adjuvants in a carrier, such as, for example, water, saline, aqueous dextrose, glycerol, ethanol and the like; solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethyl formamide; oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan; or mixtures of these substances, and the like, to thereby form a solution or suspension.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are, for example, suppositories that can be prepared by mixing the compounds described herein with, for example, suitable non-irritating excipients or carriers such as cocoa butter, polyethyleneglycol or a suppository wax, which are solid at ordinary temperatures but liquid at body temperature and therefore, melt while in a suitable body cavity and release the active component therein.

Dosage forms for topical administration may include, for example, ointments, powders, sprays, and inhalants. The active component is admixed under sterile conditions with a physiologically acceptable carrier and any preservatives, buffers, or propellants as can be required. Ophthalmic formulations, eye ointments, powders, and solutions also can be employed.

Generally, depending on the intended mode of administration, the pharmaceutically acceptable compositions will contain about 1% to about 99% by weight of the compounds described herein, or a pharmaceutically acceptable salt thereof, and 99% to 1% by weight of a pharmaceutically acceptable excipient. In one example, the composition will be between about 5% and about 75% by weight of a compounds described herein, or a pharmaceutically acceptable salt thereof, with the rest being suitable pharmaceutical excipients.

Actual methods of preparing such dosage forms are known, or will be apparent, to those skilled in this art. Reference is made, for example, to Remington's Pharmaceutical Sciences, 18th Ed., (Mack Publishing Company, Easton, Pa., 1990).

In some embodiments, the composition does not include one or more other anticancer compounds. In other embodiments, the composition includes one or more other anticancer compounds. For example, administered compositions can comprise standard of care agents for the type of tumors selected for treatment.

In another aspect, kits are provided. Kits according to the invention include package(s) comprising compounds or compositions of the invention. In one embodiment, kits comprise Compound (1), or a pharmaceutically acceptable salt thereof, and a compound selected from the group consisting of Compound (2a) and Compound (2b), or a pharmaceutically acceptable salt thereof.

The phrase “package” means any vessel containing compounds or compositions presented herein. In some embodiments, the package can be a box or wrapping. Packaging materials for use in packaging pharmaceutical products are well-known to those of skill in the art. Examples of pharmaceutical packaging materials include, but are not limited to, bottles, tubes, inhalers, pumps, bags, vials, containers, syringes, bottles, and any packaging material suitable for a selected formulation and intended mode of administration and treatment.

The kit also can contain items that are not contained within the package but are attached to the outside of the package, for example, pipettes.

Kits can contain instructions for administering compounds or compositions of the invention to a patient. Kits also can comprise instructions for approved uses of compounds herein by regulatory agencies, such as the United States Food and Drug Administration. Kits also can contain labeling or product inserts for the inventive compounds. The package(s) and/or any product insert(s) may themselves be approved by regulatory agencies. The kits can include compounds in the solid phase or in a liquid phase (such as buffers provided) in a package. The kits also can include buffers for preparing solutions for conducting the methods, and pipettes for transferring liquids from one container to another.

Examples have been set forth below for the purpose of illustration and to describe certain specific embodiments of the invention. However, the scope of the claims is not to be in any way limited by the examples set forth herein.

### Example 1

### In Vitro Activity of Compound (1) in Combination with Compound (2b)

This study describes the activity of individual anticancer agents Compound (1) and Compound (2b), as well as their combination, in a panel of 81 cancer cell lines. Cell lines were selected to represent 17 different indications with many different genetic variations and biochemical characteristics. In addition, the study included resting Peripheral Blood Mononuclear Cells, PBMC, as a model for non-proliferating cells. The results of individual activity profiles were further used to perform a combination study of Compound (1) and Compound (2b) using a panel of 81 cell lines. The study also compared the activity profiles of Compound (1) and Compound (2b) with profiles of more than 300 known anticancer agents.

Prior to in vitro combination studies, the activity of individual agents was investigated using a panel of 82 cell lines. The purpose of testing individual agents was to determine the independence of their action. In addition, comparison to an activity profile of known anticancer agents may help form a hypothesis regarding potential mechanisms of the compounds' action.

### Materials and Methods

Cell lines were purchased directly from the ATCC, NCI, CLS, and DSMZ cell line collections. A master bank and working aliquots were prepared. Cells used for the study had undergone less than 20 passages. To ensure the absence of potential contamination and wrong assignment, all cell lines were tested on the Whole Genome Array (Agilent, USA) and by STR analysis. Absence of mycoplasma and SMRV contamination was confirmed for all cell lines used in the studies.

The cell lines were grown in the media recommended by the suppliers in the presence of 100 U/ml penicillin G and 100 μg/ml streptomycin supplied with 10% FCS (PAN, Germany). The RPMI 1640, DMEM, and MEM Earle's medium were from Lonza (Cologne, Germany), supplements 2 mM L-glutamine, 1 mM Na-pyruvate and 1% NEAA were from PAN (Aidenbach, Germany), 2.5% horse serum and 1 unit/ml insulin from Sigma-Aldrich (Munich, Germany). RPMI medium was used for culturing the following cell lines: 5637, 22RV1, 7860, A2780, A431, A549, ACHN, ASPC1, BT20, BXPC3, CAKI1, CLS439, COLO205, COLO678, DLD1, DU145, EFO21, EJ28, HCT15, HS578T, IGROV1, JAR, LOVO, MCF7, MDAMB231, MDAMB435, MDAMB436, MDAMB468, MHHES1, MT3, NCIH292, NCIH358M, NCIH460, NCIH82, OVCAR3, OVCAR4, PANC1005 (addition of insulin), PBMC, PC3, RDES, SF268, SF295, SKBR3, SKMEL28, SKMEL5, SKOV3, SW620, U2O5, UMUC3, and UO31.

DMEM was used for A204, A375, A673, C33A, CASKI, HCT116, HEPG2, HS729, HT29, J82, MG63, MIAPACA2 (addition of horse serum), PANC1, PLCPRF5, RD, SAOS2, SKLMS1, SKNAS, SNB75, T24, and TE671.

MEM Earle's medium was used for CACO2, CALU6, HEK293, HELA, HT1080, IMR90, JEG3, JIMT1, SKHEP1, SKNSH, and U87MG.

Cells were grown in 5% CO2 atmosphere in a HeraCell 150 incubator (Thermo Scientific, Germany).

The following is a list of compounds used in the studies:

| | | | Concentration | |
|---|---|---|---|---|
| | | | of a stock | |
| | | | solution | |
| Container | Amount | Dissolved | (max. final | |
| bar code | supplied | in | concentration) | Supplier |
| Compound | 10.27 mg | 439 μl | 50 mM | EMD Serono |
| (1) | | DMSO | (50 μM) | (Rockland MA, |
| | | | | USA) |
| Compound | 10.3 mg | 762 μl | 50 mM | EMD Serono |
| (2b) | | DMSO | (50 μM) | (Rockland MA, |
| | | | | USA) |
| 5-FU | NA | DMSO | 100 mM | Lot #22808088 |
| | | | (100 μM) | (Sigma-Aldrich) |
| Paclitaxel | NA | DMSO | 10 mM | Lot#ASM-110 |
| | | | (10 μM) | (LC |
| | | | | Laboratories) |

The stock solutions of Compound (1) and Compound (2b) were prepared in DMSO (Sigma-Aldrich, Germany) as indicated in table above. Stock solutions were further aliquoted and stored under argon at −20° C.

10% w/v of trichloracetic acid, TCA (Sigma-Aldrich, Germany), was prepared in distilled water. 0.08% wt/v sulforhodamine B, SRB (Sigma-Aldrich, Germany) solution was prepared in 1% acetic acid (Sigma-Aldrich). Tris base was purchased from Karl Roth (Germany).

Cell growth and treatment were performed in 96-well microtitre plates CELLSTAR® (Greiner Bio-One, Germany). Cells harvested from exponential phase cultures by trypsinization were plated in 150 μl of media at optimal seeding densities. The optimal seeding densities for each cell line were determined to ensure exponential growth for the duration of the experiment. All cells growing without anticancer agents were sub-confluent by the end of the treatment as determined by visual inspection.

Compound dilutions in DMSO were performed in 96-well rigid PCR plates. Compounds were then diluted 1:250 in RPMI medium.

150 μl of cells, after a 24-hour pre-growth period, were treated by mixing with 50 μl of the compound containing media (resulting in a final DMSO concentration of 0.1%). The cells were allowed to grow at 37° C. for 72 hours. In addition, all experiments contained a few plates with cells that were processed for measurement immediately after the 24 hours recovery period. These plates contained information about the cell number that existed before treatment, at time zero, and served to calculate the cytotoxicity.

After treatment, cells were precipitated by addition of 10% TCA. Prior to fixation, the media was aspirated as described. After an hour of incubation at 4° C., the plates were washed two times with 400 μl of deionized water. Cells were then stained with 100 μl of a 0.08% wt/v SRB. The plates were allowed to sit for at least 30 min. and washed six times with 1% acetic acid to remove unbound stain. The plates were left to dry at room temperature and bound SRB was solubilized with 100 μl of 10 mM Tris base. Measurement of optical density was performed at 560 nm on a Victor 2 plate reader (Perkin Elmer, Germany). The SRB values for A375 and H460 cell lines were near to saturation (2.5 OD units) due to the high protein content of these cells, but not cell confluence. The measurements for these cells were performed at 520 nm instead of 560 nm.

Prior to in vitro combination studies, the activity of individual agents was investigated using a panel of 80 cell lines. The purpose of testing individual agents was to determine the independence of their action. In addition, comparison to an activity profile of known anticancer agents may help form a hypothesis regarding potential mechanisms of the compounds' action.

The calculations used nomenclature introduced by DTP NCI. Unprocessed optical density data from each microtitre plate were stored in MS Excel or as a text file in a databank. The first step of data processing was calculating an average background value for each plate, derived from wells containing medium without cells. The average background optical density was then subtracted from the appropriate control values (containing cells without addition of a drug), from values representing the cells treated with an anticancer agent, and from values of wells containing cells at time zero. Thus the following values were obtained for each experiment: control cell growth, C; cells in the presence of an anticancer agent T, and cells prior to compound treatment at time zero, T_{z} (or T₀, in some publications).

The Z-factor is a parameter commonly used to assess quality of the assay performance and was calculated according to the following equation:

$${Z}^{′} = 1 - \frac{( 3 {σ}_{c +} + 3 {σ}_{c -} )}{{μ}_{c +} - {μ}_{c -}}$$

where μ_{c+} and μ_{c-} are denoted for the means of positive and negative control signals and σ_{c+} and σ_{c-} are their standard deviation. In a way, the Z′-factor reflects the significance of the dynamic range of the measurements recorded and should be >0.5. In this study, Z′-factor was applied to determine the significance of signals over background for T_{z} and C values. The results of the screening were accepted only if the Z-factor was above 0.5 for each case.

The non-linear curve fitting calculations were performed using in-house developed algorithms and visualization tools. The algorithms are similar to those previously described and were complemented with the mean square error or MSE model. This can be compared to commercial applications, e.g. XLfit (ID Business Solutions Ltd., Guild-ford, UK) algorithm “205”. The calculations included the dose response curves with the best approximation line, a 95% confidence interval for the 50% effect (see below).

A common way to express the effect of an anticancer agent is to measure cell viability and survival in the presence of the test agent as % T/C×100. The relationship between viability and dose is called a dose response curve. Two major values are used to describe this relationship without needing to show the curve: the concentration of test agents giving a % T/C value of 50%, or 50% growth inhibition (IC₅₀), and a % T/C value of 10%, or 90% growth inhibition (IC₉₀).

Using these measurements, cellular responses can be calculated for incomplete inhibition of cell growth (GI), complete inhibition of cell growth (T GI) and net loss of cells (LC) due to compound activity. Growth inhibition of 50% (GI₅₀) is calculated as 100×[(Tᵢ−T_{z})/(C−T_{z})]=50. This is the drug concentration causing a 50% reduction compared to the net protein increase in control cells during the drug incubation period. In other words, GI₅₀ is IC₅₀ corrected for time zero. Similar to IC₉₀, calculated GI₉₀ values are also reported for all compounds tested. TGI was calculated from Tᵢ=T_{z}. LC₅₀ is the concentration of drug causing a 50% reduction in the measured protein at the end of the drug incubation period compared to that at the beginning. It was calculated as 100×[(Tᵢ−T_{z})/T_{z}]=−50. However, due to 72 hours treatment, low cell seeding density was required and LC₅₀ could rarely be achieved.

The IC₅₀, IC₉₀, GI₅₀, GI₉₀ and T GI values were computed automatically. Visual analysis of all dose response curves was performed to check the quality of the fitting algorithm. In cases where the effect was not reached or exceeded, the values were either approximated or expressed as “-”. In this study all values were greater than the maximum drug concentration tested. In these cases, the values were either excluded from the analysis, or approximation of IC₁₀ and GI₁₀ were used for analysis.

All values were log 10-transformed for analysis. This transformation ensures better data fitting to the normal distribution, a prerequisite to apply any statistical tool. Statistical analyzes were performed using proprietary software developed at Oncolead integrated as a database analysis tool. However, except for database comparison, the analysis can be reproduced using either MS Excel or STATISTICA® (StatSoft, Hamburg). Using MS Excel: identification of mean, e.g. mean GI₅₀ (function: “Average”); calculation of ,δ, delta (GI₅₀−mean GI₅₀); and z-score (function “Standardize”). Comparison of the activity profile of Compound (1) and Compound (2b) cross-correlation could be performed using Pearson and Spearman correlations (for example by using STATISITCA®). In addition, Pearson pairwise and Spearman pairwise comparisons were used to increase the confidence of the results. Pairwise comparison was calculated based on pairwise similarity of the agents to all tested agents in the database.

Z-score is a way to report standard deviations rather than absolute deltas and mean values. It indicates how far the value deviated from its mean in units of standard deviation:

$$Z = \frac{X - μ}{{σ}_{x}} = \frac{δ}{{σ}_{x}}$$

where X is a single measured value, e.g. GI₅₀, and μ is a mean of all measured values (mean GI₅₀) and σₓ is a standard deviation of X.

The concept of the mean graph introduced by NCI permits visualization of a cell activity parameter for a given anticancer drug in all cells. This graph yields a characteristic pattern that provides rich information for visual comparison. The values are plotted as horizontal bars from the mean values. Each bar, therefore, represents the relative activity of the compound in the given cell lines deviating from the mean in all cell lines. In contrast to NCI, z-score values were plotted rather than absolute delta. In statistical terms, z-values represent a standard deviation that provides a kind of normalization and simplifies comparison between compounds with different activity distributions. In addition, an averaged combined z-score was calculated for cell lines of the same origin.

Z-score values as well as the range of tested concentrations were included in all visualizations. The applicability of z-score graphs should be considered with precaution if the agent's activity does not follow the normal distribution.

The most sensitive and non-sensitive cell lines were visualized by using either a box-plot graph or by selecting the eight most and least sensitive cell lines using the z-score for each agent. This also applied to the cell lines where activity of an agent could not be determined. Box plots were constructed from five values: the smallest value (the lowest whisker), the first quartile (the lowest border of the box), the median (square in the middle), the third quartile (the upper border of the box), and the largest value (the highest whisker).

The screening was designed to determine potential synergistic combinations. All and/or part of the 5×5 or 7×7 matrix were used to design the study. Bliss independence was used as a basis for calculations, unless otherwise stated. The following parameters were calculated:

δᵢ=Measured valueᵢ−Theoretical valueᵢ

where i=[1 . . . n] is one of the values of the matrix used and theoretical value, calculated as described for the Bliss Independence method. Vector sum was determined as:

$$Vector sum = {\sum }_{i = 1}^{n} Sign ( {Effect}_{i} ) {Effect}_{i}^{2}$$

in this term the Vector Sum rather represents scalar:

$$Vector sum average = \frac{1}{n} {\sum }_{i = 1}^{n} {Effect}_{i} = Mean ( {Effect}_{i} )$$

The average values below—0.5 indicate a strong synergy effect: (−0.5, −0.02)—Synergy effect, (−0.2, 0.02)—Zero effect (additivism), (0.02, 0.5)—potential antagonism, and above 0.5—strong antagonism. However, it is possible that the effect of the combination is not synergistic (or even antagonistic) but still better than each of the agents alone. Moreover, in vivo, any effect better than a single agent is considered clinically positive (or synergistic). In this case, one considers a potential interaction of two agents that can be determined by the highest single agent, HSA, model. This model determines the difference between the larger effects produced by one of the single agents at the same concentrations as in the mixture.

Single Bestᵢ=Best of [Agent 1ᵢ:agent 2ᵢ]

and delta HSA, for two agents can be determined as:

$$\begin{matrix}{deltaHSA}_{i} = δ {HSA}_{i} \\ = {MeasuredValue}_{i} - {SingleBest}_{i}\end{matrix}$$

$$and$$

$$AverageHSAEffect = \frac{{\sum }_{i = 1}^{n} δ {HSA}_{i}}{n}$$

### Summary of In Vitro Results

Efficacy of Compound (1) varies broadly from 4-5 nM in sensitive cell lines to minimal activity at 50 μM in the most non-sensitive cell lines. Under the conditions tested, minimal activity could be determined for cancer cell lines: A673, HEK293, J82, JAR, JEG3, MDAMB436, MDAMB468, MHHES1, NCIH82, PANC1, PLCPRF5, and SF268. For cell lines CLS439, EFO2,1 PC3, SAOS2, SF295, and SKOV3, activity was estimated above the highest tested concentration of 50 μM. At the same time, 50% of the cell lines tested exhibited a sensitivity below 500 nM (the median is 490 nM), and 27 of 82 cell lines were found to be sensitive below 100 nM of Compound (1). Action of Compound (1) and Compound (2b) was synergistic in a larger number of human cancer cell lines, which suggests that the mechanisms of compound action are complementary. A673 cells are non-sensitive to the action of Compound (1) or Compound (2b) alone, but can show strong synergy in combination. A549 and MCF7 cells show some sensitivity to both agents, which can be further potentiated with their combination. SKBR3 cell line is very sensitive to Compound (2b). However, the effect can be further increased by the combination of both agents. These findings may be related to the all breast cancer cell lines with overexpression of the HER2 gene.

The most sensitive cell lines were HT29, COLO205, TE671, A375, SKMEL5, COLO678, SKNAS, and NCIH292, where Compound (1) showed activity between 4.8 and 8 nM. The difference between the most and least sensitive cell lines was as large as 10.000-fold. Due to such a large window of activity, the activity distribution is broad and does not follow a normal distribution. In such a case, z-score has little statistical meaning; however, it can still be applicable, for example, to group activities according to therapeutic indications.

The rank of Compound (1) activity (or rank of z-score values) is another tool that can be applied. These properties of Compound (1) stress the necessity of using diverse analysis tools and covering a broad concentration range to test anticancer agents. One possibility is that Compound (1) has a specific mechanism of action and acts only on a sub-population of tumor cells.

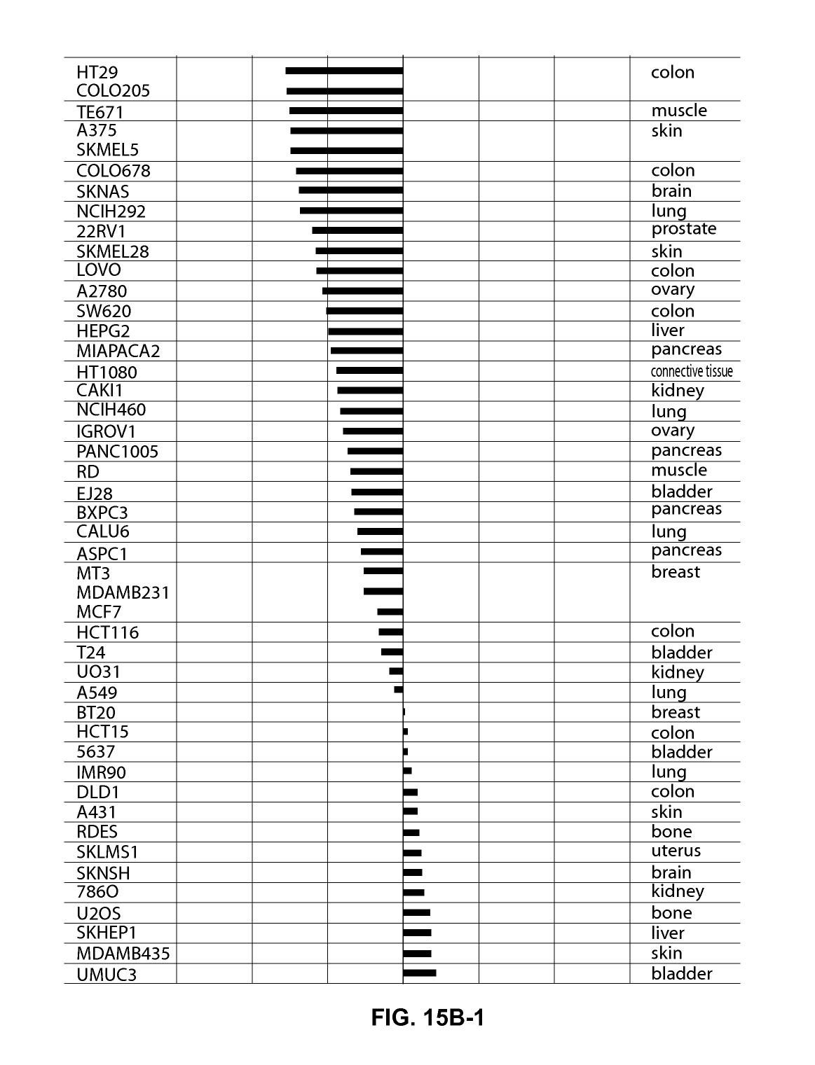

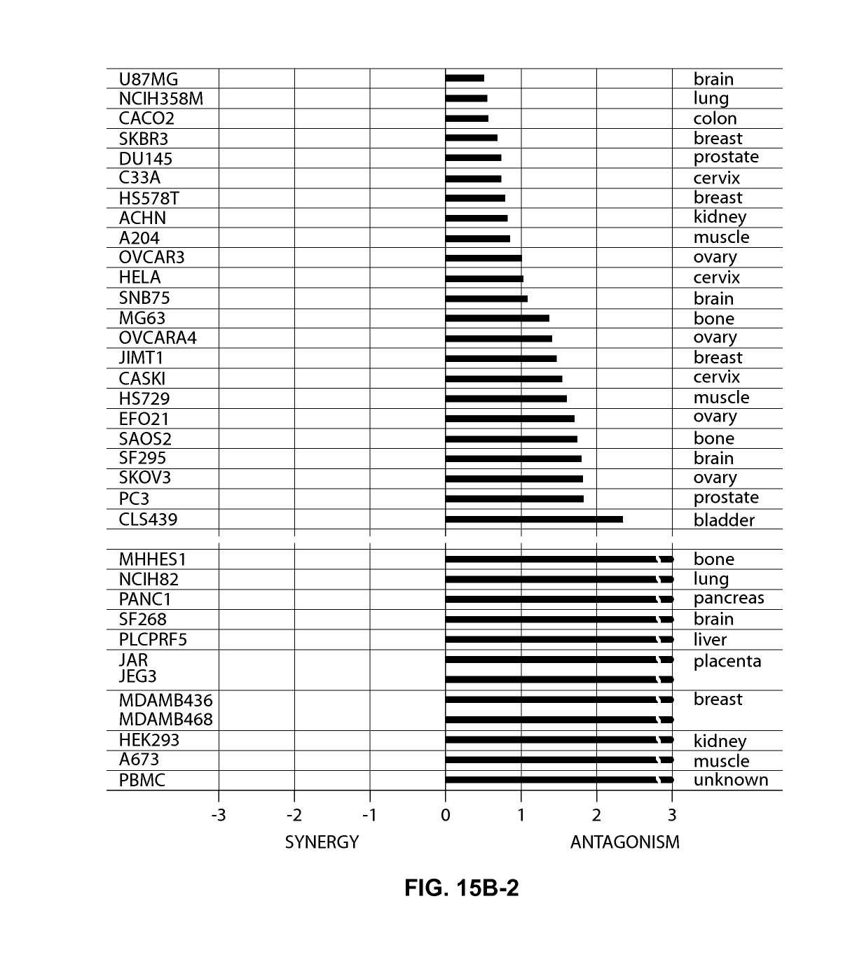

The 81 human cancer cell lines represented 17 different tumor origins. FIGS. 15A and 15B show individual z-scores within one tumor origin group, as well as combined z-scores for each therapeutic indication as an average value (green triangle). As in the case of individual z-scores, direction to the left points towards sensitivity to the compound action. A zeroline corresponds to average activity. The data suggest that lung, pancreas, colon, and melanoma cell lines are generally more sensitive to Compound (1), since the average value of z-scores are on the left. All but one pancreas (PANC1) cell line are very sensitive to Compound (1) action. HT1080 is also a very sensitive cell line.

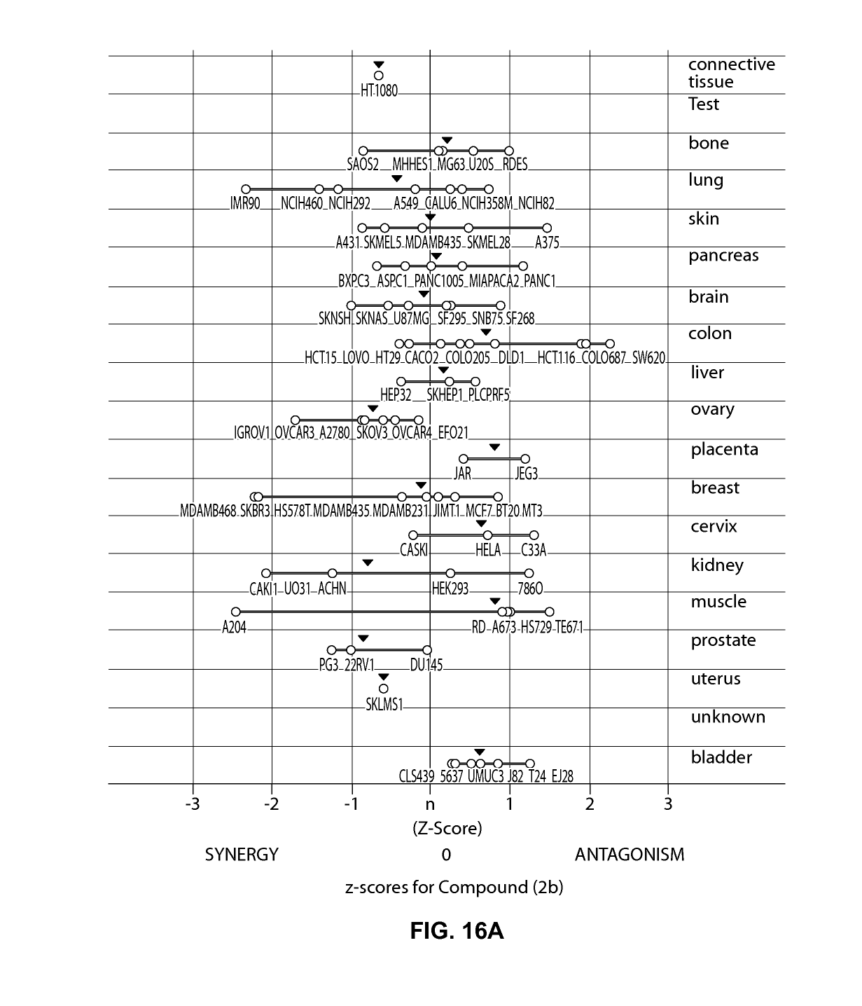

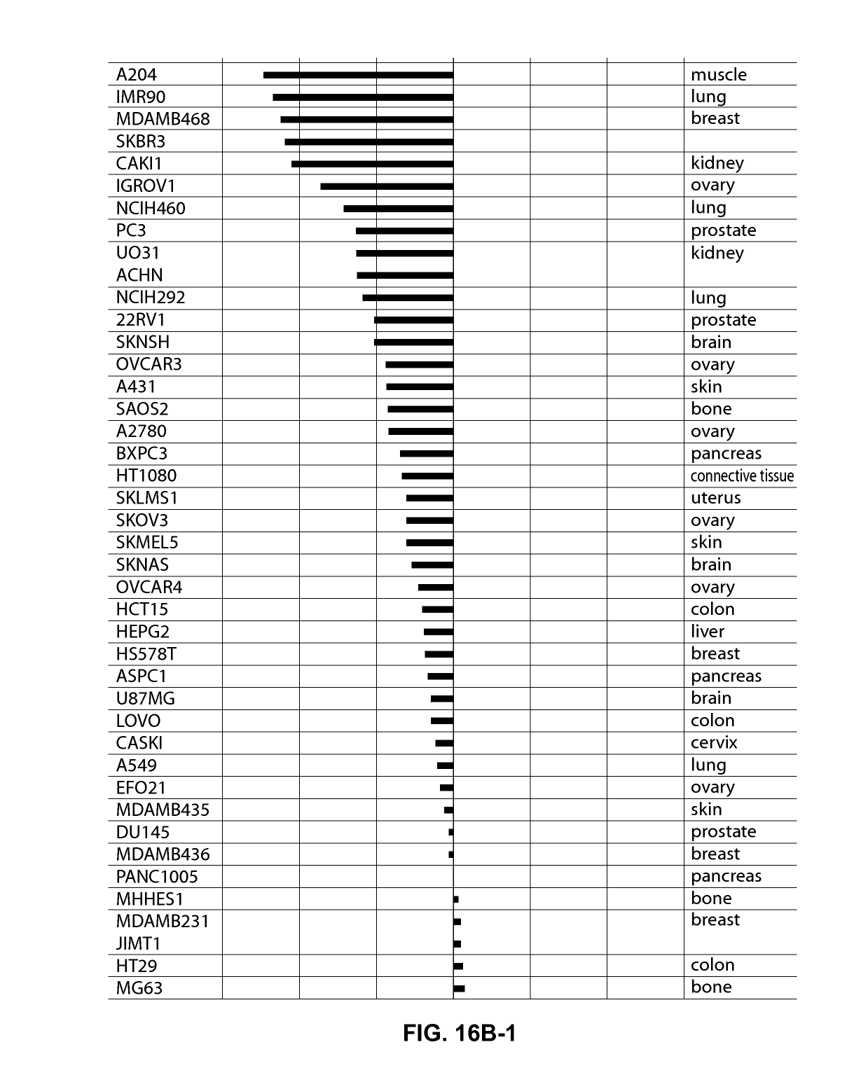

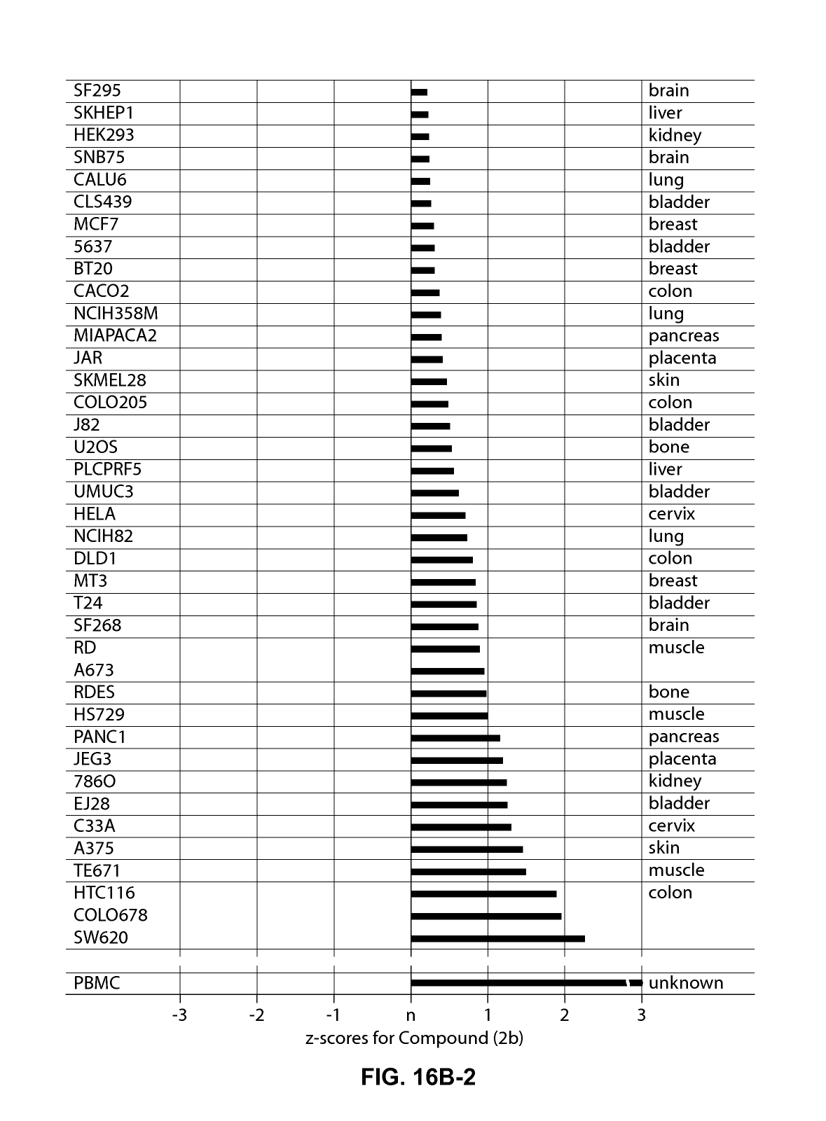

Activity, GI₅₀ values, of Compound (2b) in cell lines ranged between <500 nM in A204, IMR90, MDAMB468, SKBR3, CAKI1, and IGROV1 (most sensitive, as determined by z-score <−1.5) and >4 μM in SW620, COLO678, and HCT116 (non-sensitive cell lines, z score >1.5). These results may indicate that cell lines showing the strongest negative deviation of z-scores from the mean will also show activity in other biological systems, e.g., mouse xenograft models. The average GI₅₀ value in all 81 cell lines was 1.3-1.4 μM, calculated based on log 10-transformed data. No activity was shown in arrested PBMC suggesting that Compound (2b) may act preferably on proliferating cells. FIGS. 16A and 16B show that the activity distribution is narrow, but sensitive cell lines can be well-discriminated.

Comparison of the Compound (2b) activity profile with an internal databank containing more than 300 different anticancer agents identified a number of agents. The most similar agent (average similarity above 0.8) is MSC2208382A. Weaker similarity (above 0.7) is detected with GDC-0941 bismesylate and ZSTK474, and some degree of similarity to MSC2313080A. GDC-0941 bismesylate is an analog of PI-103, a dual PI3K/mTOR inhibitor and considered to be a relatively specific inhibitor of class I PI3K enzymes as well as ZSTK474. It could be suggested that Compound (2b) belongs to the class of PI3K inhibitors.

As in the case of individual z-scores, the direction to the left points towards sensitivity to the compound action. A zero-line corresponds to average activity. Ovarian and prostate tumors could be specific therapeutic areas. At least for all cell lines tested, the z-score is below zero. Applications for breast, lung, and renal tumors also could be considered. However, each of the indications contains cell lines either very sensitive or non-sensitive to Compound (2b) action.

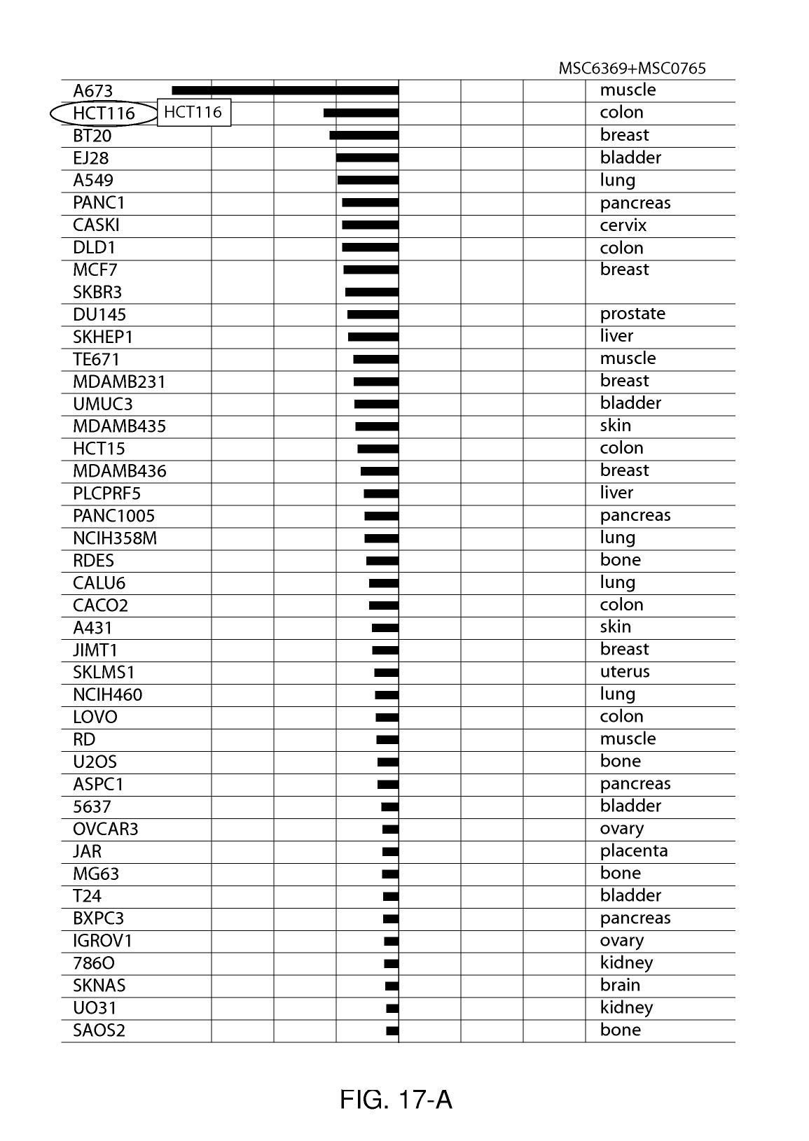

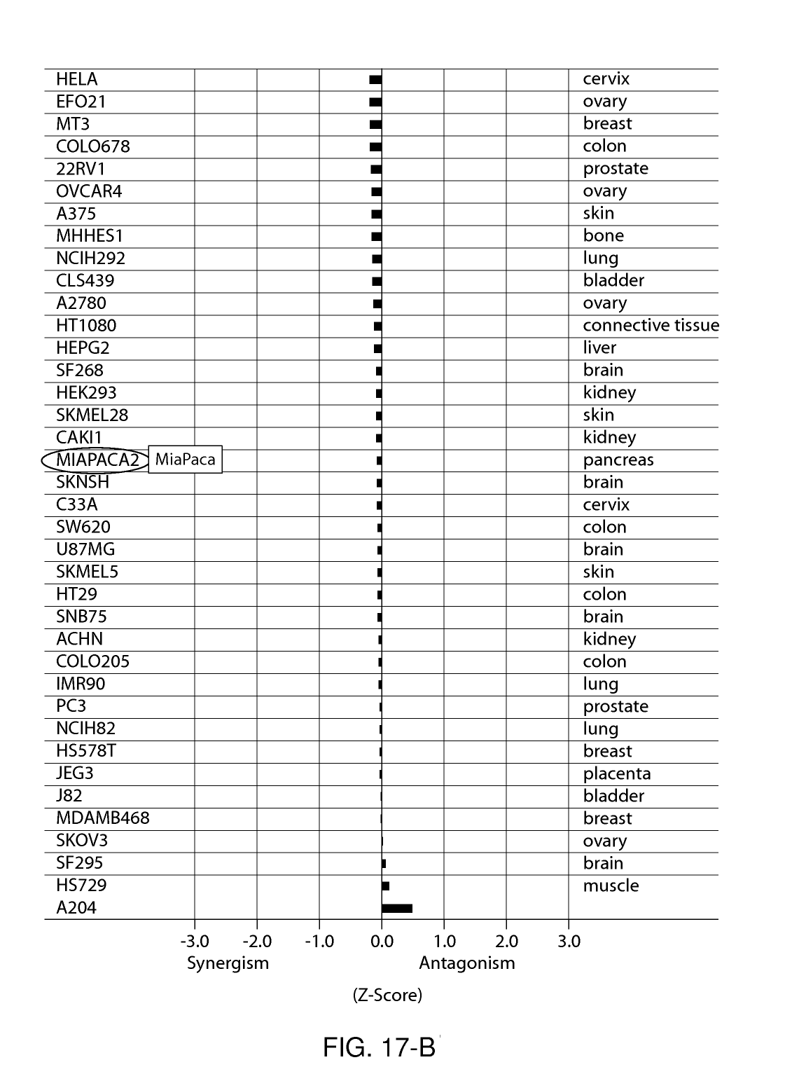

Although most of the cell lines showed potential synergy for in vitro combination of Compound (1) and Compound (2b), the results with a vector sum of below −1 can be considered significant. Table 1 and FIG. 17 summarize the results. Cell line A673 is non-sensitive to the action of Compound (1) or Compound (2b) alone, but shows strong synergy in combination. However, from in vivo or clinical perspectives, cell line groups four and five are probably more relevant. Activity (GI₅₀) of Compound (1) is 300 nM and 150 nM in A549 and MCF7 cells, respectively, which is comparable with 100 nM activity in the most sensitive cell lines. Activity (GI₅₀) of Compound (2b) is 1.15 μM and 1.6 μM in A549 and MCF7 cells, respectively, below or close to the average activity of 1.3-1.4 μM for this agent. The combination index for these cell lines is close to −1, which is indicative of synergy. Another example is SKBR3. This cell line is very sensitive to Compound (2b) and non-sensitive to Compound (1). However, the effect can be further increased by the combination of both agents.

Compound (1) and Compound (2b) act on proliferating cells and showed no activity in resting PBMC. However, these agents differ in their activity. The difference between the most and least sensitive cell lines for Compound (1) was as large as 10.000-fold. For the most insensitive cell lines, resistance extends beyond the tested concentration range >50 μM.

Thus, it appears that Compound (1) may have a specific mechanism of action and acts only on a sub-population of tumor cells. Selection of therapeutic indications in the clinic can be complemented by the mutational analysis. In contrast, Compound (2b) shows narrow activity in cell lines. The separation between sensitive and insensitive cell lines is statistically significant but the differences in activity are in the range of 10-20-fold. The activity profile of Compound (2b) has similarities to the PI3K inhibitors, e.g. PI-103 or its pharmalog GDC-0941. No prediction could be made about the agent's activity and the mutational status of genes involved in activation of the PI3K pathway, e.g. EGFR, PTEN, and PI3K. Some markers may be predictive for induction of apoptosis upon action of this PI3K inhibitor: EGFR (mutation), HER2 (amplification), MET (mutation/amplification). Indirectly, this fact can be supported by the observation that SKBR3 cells (HER2 amplification) were among the most sensitive cell lines.

Compound (1) and Compound (2b) were further tested in combination in all cell lines using a 7×7 matrix, with variation around GI₅₀ averaged in all cell lines for each of the agents. The rationale for selecting this concentration was as follows. First, this concentration is a reference concentration that describes efficacy of the anticancer agents in cellular models, i.e. only cell lines that show significant effects below mean GI₅₀. Second, it is known that efficacy of anticancer agents is limited, based on citations reporting 10-30%. Therefore, selection of mean GI₅₀ would correspond to the expected efficacy of approximately 50%. Third, the variation spanned by the 7×7 matrix (almost ten-fold in both directions from the mean GI50) allows enough coverage to address the question of whether there are any potential interactions between the two agents.

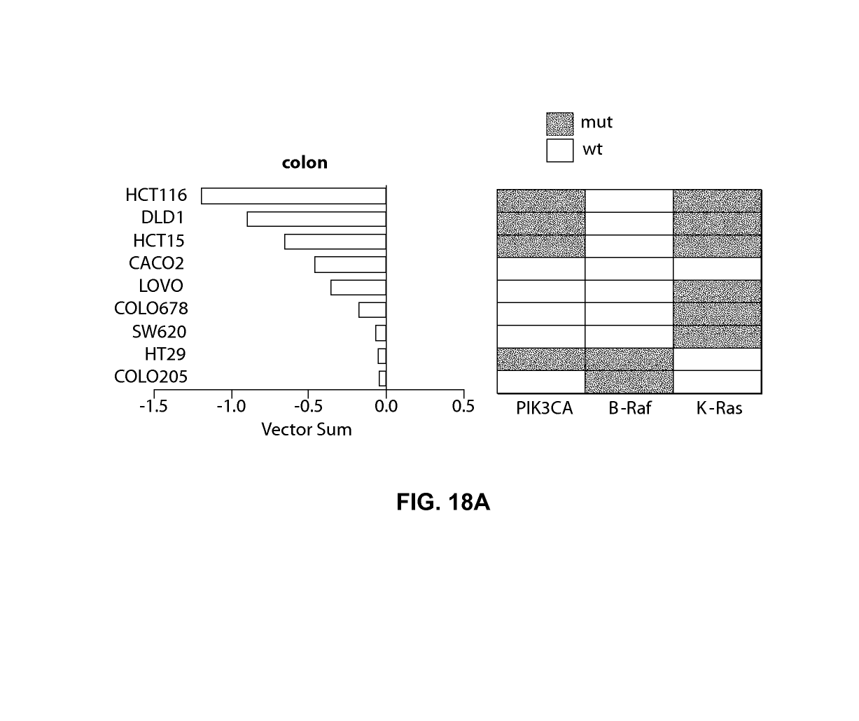

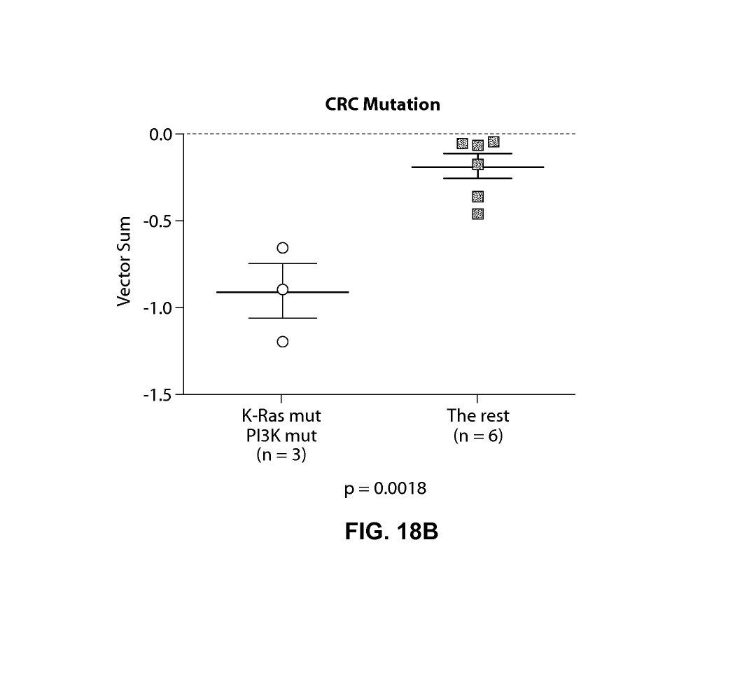

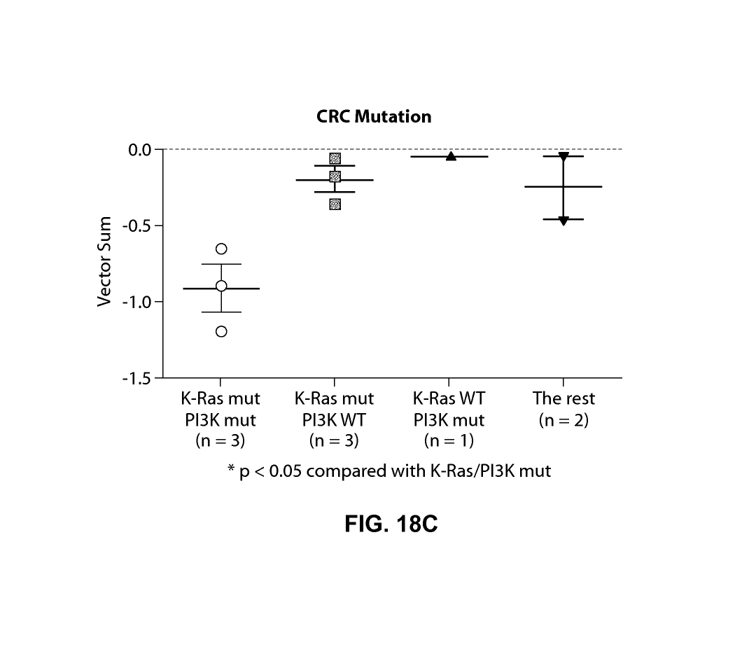

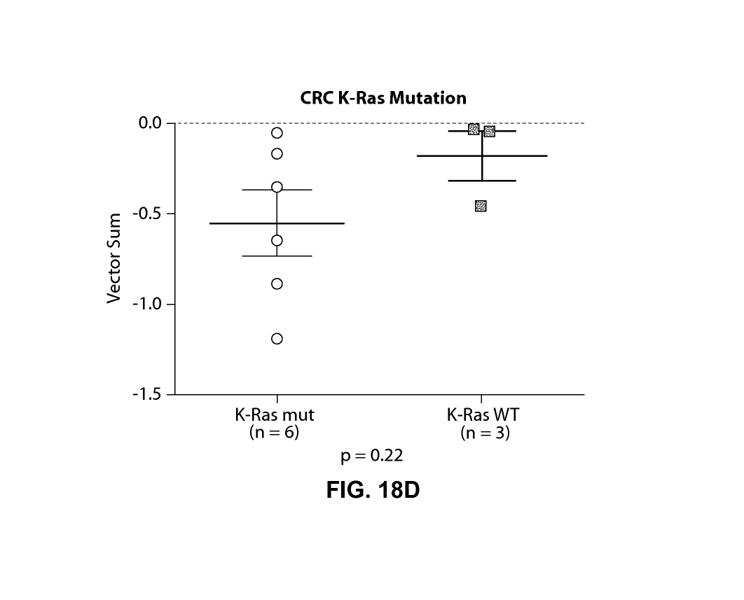

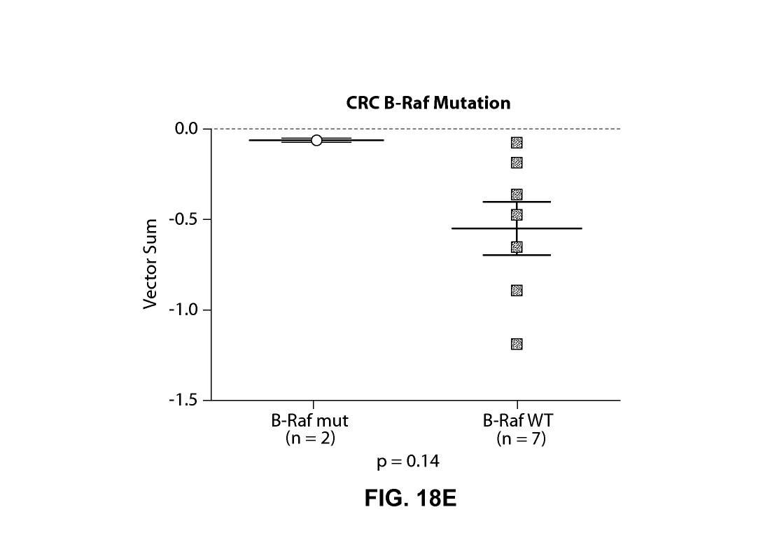

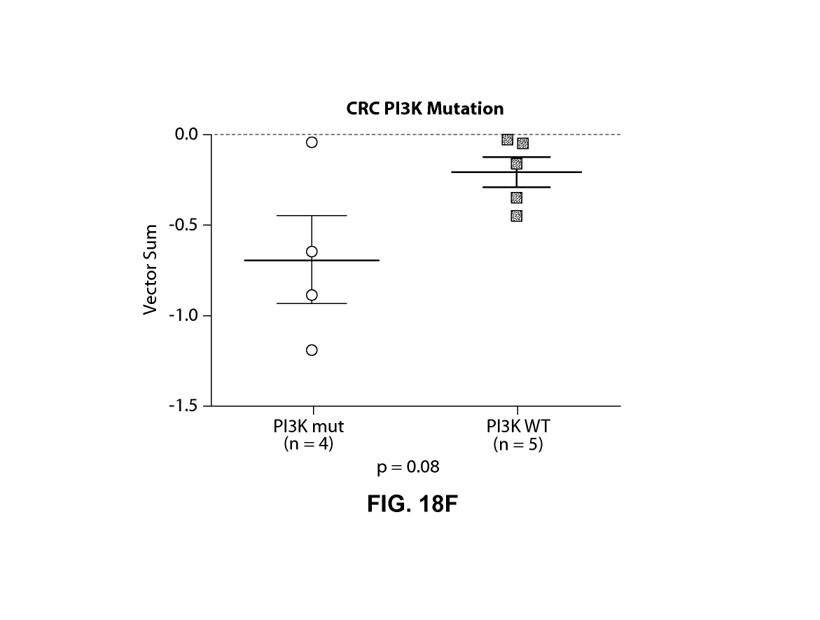

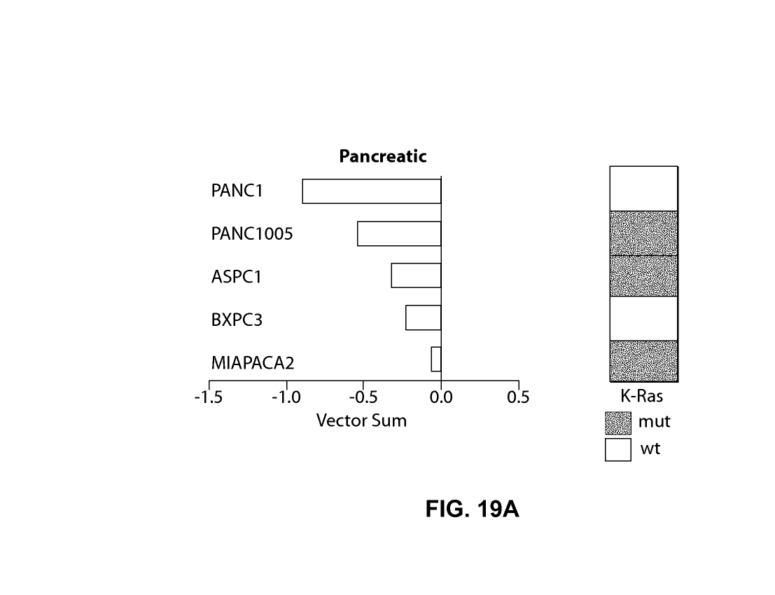

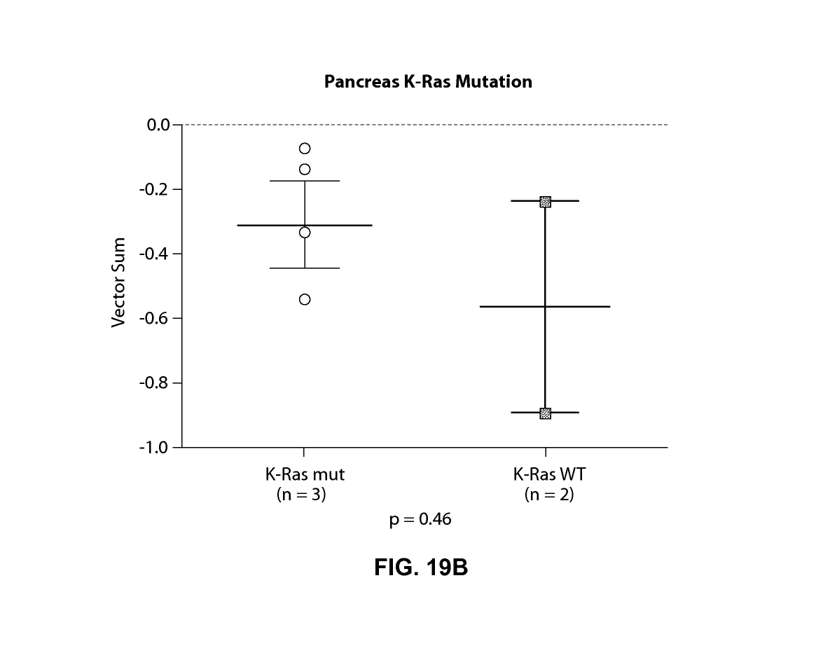

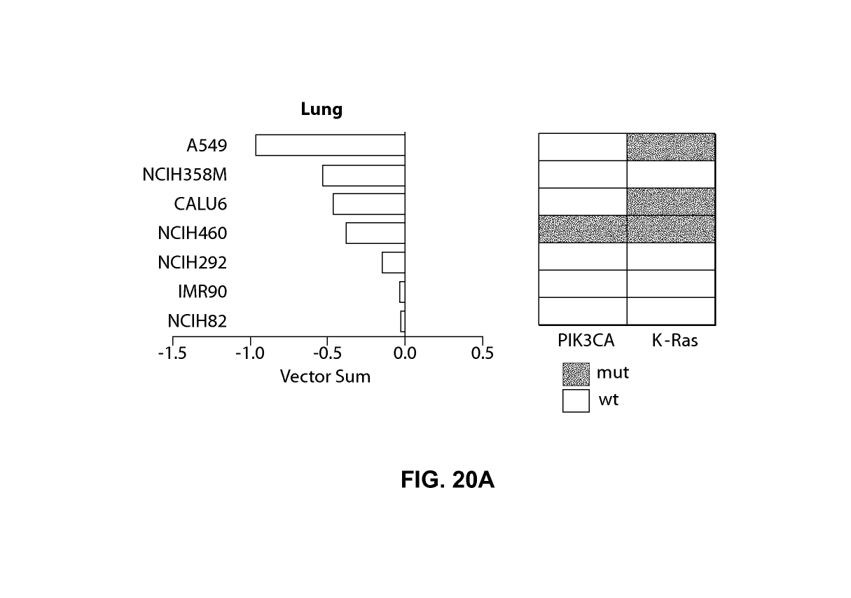

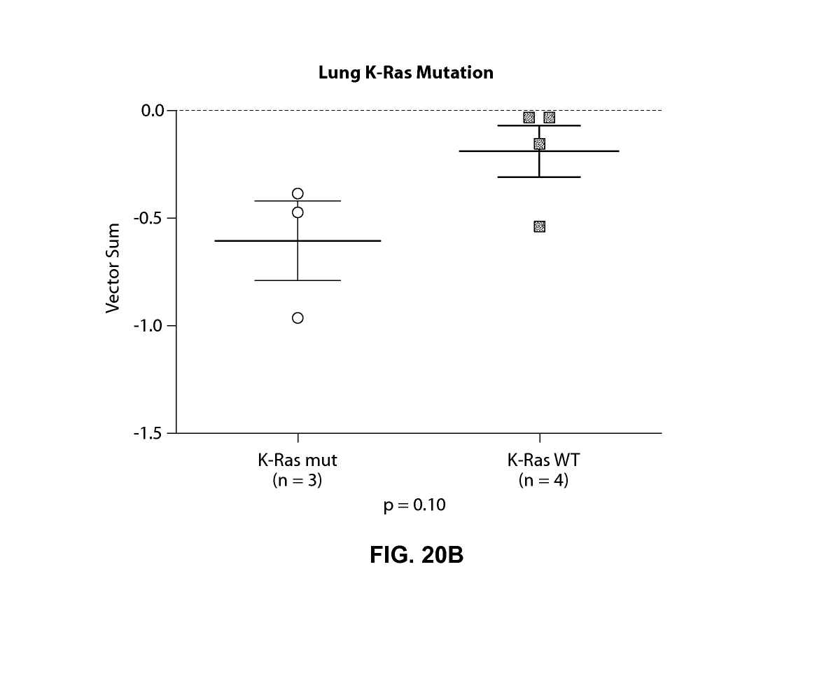

In almost all cases, Compound (1) and Compound (2b) in combination showed potential to be synergistic (FIG. 17), as determined by the Bliss Independence model (see, for example, Yan et al., BMC Systems Biology, 4:50 (2010)). See also FIGS. 18A, 18B, 18C, 18D, 18E, 18F, 19A, 19B, 20A, 20B.

However, the strongest synergistic effect was detected when the activity of either agent was weak. This may be attributed, at least in part, to experimental set-up, i.e., any effect of combination is considered significant if the agents alone mediate little, if any effect on the cells. Alternatively, the effect of a single agent can be too strong to detect increasing effects. In the later case, the HSA model provides a better view of the potential interaction between two agents.

### Example 2

### In Vivo Activity of Compound (1) in Combination with Compound (2b) or Compound (2a) Against Subcutaneous Human Colon Carcinoma HCT 116 Bearing SCID Mice

To evaluate the antitumor activity of the MEK inhibitor Compound (1) in combination with the pan-PI3K inhibitor Compound (2a) or the dual pan-PI3K/mTOR inhibitor Compound (2b), experiments were conducted using female SCID mice bearing human colon carcinoma HCT 116 (KRAS and PIK3CA mutant) xenografts. Four studies were performed:

In a first study, a low dose of Compound (1) at 5 mg/kg was tested in combination with Compound (2b) at 30 mg/kg and Compound (2a) at 50 and 75 mg/kg.

In a second study, the dose of Compound (1) was increased to 10 and 20 mg/kg in combination with Compound (2b) at 20 mg/kg, and Compound (1) at 10 mg/kg was combined with Compound (2a) at 50 and 75 mg/kg.

In a third study, used as a confirmation study, the dose of Compound (1) was used at 10 and 20 mg/kg in combination with Compound (2a) at 50 and 75 mg/kg.

In a fourth study, used as a confirmation study, the dose of Compound (1) was used at 10 and 20 mg/kg in combination with Compound (2b) at 20 mg/kg.

### Materials and Methods

CB17/1CR-Prkdc severe combined immunodeficiency (SCID)/Crl mice, at 8-10 weeks old, were bred at Charles River France (Domaine des Oncins, 69210 L'Arbresle, France) from strains obtained from Charles River, USA. Mice were over 18 g at start of treatment after an acclimatization time of at least 5 days. The mice had free access to food (UAR reference 113, Villemoisson, 91160 Epinay sur Orge, France) and sterile water. The mice were housed on a 12 hours light/dark cycle. Environmental conditions including animal maintenance, room temperature (22° C.±2° C.), relative humidity (55%±15%) and lighting times were recorded by the supervisor of laboratory animal sciences and welfare (LASW) and archived.

Human colon carcinoma HCT 116 cells were purchased at American Type Culture Collection [(ATCC), Rockville, Md., USA). The HCT 116 cells were cultured in Dulbecco's modified Eagle's medium (DMEM) (Invitrogen). The tumor model was established by implanting (SC) 3×10⁶ cells mixed with 50% matrigel (Reference 356234, Becton Dickinson Biosciences) per SCID female mice.

Compound (1) formulation was prepared by incorporating the MEK inhibitor into 0.5% CMC 0.25% Tween 20. The preparation was stored at 4° C. and resuspended by vortexing before use. The oral form of the compound was prepared every 3 days. The volume of administration per mouse was 10 mL/kg.

Compound (2a) formulation was prepared in water for injection. The stock solution was chemically stable 7 days in the dark at 4° C. The volume of administration per mouse was 10 mL/kg.

Compound (2b) formulation was prepared in 1N HCl and water for injection followed by five cycles of vortexing and sonicating. The pH of the final solution was 3. The stock solution was chemically stable 7 days in the dark at 4° C. The volume of PO administration per mouse was 10 mL/kg.

For subcutaneous implantation of tumor cells, skin in the flank of the mice was disinfected using alcohol or Betadine® solution (Alcyon) and a suspension of tumor cells was inoculated SC unilaterally under a volume of 0.2 mL using a 23 G needle.

The activity on tumor growth of Compound (1), Compound (2a) and Compound (2b) used as single agent or in combination was evaluated in four different studies. The dosages and schedule of administration for each study are described in the results section and detailed in the tables that follow.

The animals required to begin a given experiment were pooled and implanted monolaterally on day 0. Treatments were administered on measurable tumors. The solid tumors were allowed to grow to the desired volume range (animals with tumors not in the desired range were excluded). The mice were then pooled and unselectively distributed to the various treatment and control groups. Treatment started 11 days post HCT 116 tumor cell implantation as indicated in the results section and in each table. The dosages are expressed in mg/kg, based on the body weight at start of therapy. Mice were checked daily, and adverse clinical reactions noted. Each group of mice was weighed as a whole daily until the weight nadir was reached. Then, groups were weighed once to thrice weekly until the end of the experiment. Tumors were measured with a caliper 2 to 3 times weekly until final sacrifice for sampling time, tumor reached 2000 mm³ or until the animal died (whichever comes first). Solid tumor volumes were estimated from two-dimensional tumor measurements and calculated according to the following equation:

Tumor weight(mg)=Length(mm)×Width²(mm²)/2

The day of death was recorded. Surviving animals were sacrificed and macroscopic examination of the thoracic and abdominal cavities was performed.

A dosage producing a 15% body weight loss (BWL) during three consecutive days (mean of group), 20% BWL during 1 day or 10% or more drug deaths was considered an excessively toxic dosage. Animal body weights included the tumor weight.

The primary efficacy end points are ΔT/ΔC, percent median regression, partial and complete regressions (PR and CR).

Changes in tumor volume for each treated (T) and control (C) group were calculated for each tumor by subtracting the tumor volume on the day of first treatment (staging day) from the tumor volume on the specified observation day. The median ΔT is calculated for the treated group, and the median ΔC is calculated for the control group. Then the ratio ΔT/ΔC is calculated and expressed as a percentage. The dose is considered as therapeutically active when ΔT/ΔC is lower than 40% and very active when ΔT/ΔC is lower than 10%. If ΔT/ΔC is equal to or lower than 0, the dose is considered as highly active and the percentage of regression is dated.

The percent of tumor regression is defined as the % of tumor volume decrease in the treated group at a specified observation day compared to its volume on the first day of treatment. At a specific time point and for each animal, % regression is calculated. The median % regression is then calculated for the group using the following equation:

% regression(at t)=(volume at t₀−volume at t)/volume at t₀)×100

Partial regression: Regressions are defined as partial if the tumor volume decreases to 50% of the tumor volume at the start of treatment.

Complete regression: The CR is achieved when tumor volume=0 mm³ (CR is considered when tumor volume cannot be recorded).

The term “therapeutic synergy” is used when the combination of two products at given doses is more efficacious than the best of the two products alone considering the same doses. In order to study therapeutic synergy, each combination was compared to the best single agent using estimates obtained from a two-way analysis of variance with repeated measurements (Time factor) on parameter tumor volume.

Statistical analyses were performed on SAS system release 8.2 for SUN4 via Everstat V5 software and SAS 9.2 software. A probability less than 5% (p<0.05) was considered as significant.

### Results of In Vivo Studies

First Study: Antitumor Activity of Compound (1) (5 mg/kg) in Combination with Compound (2b) (30 mg/kg) or Compound (2a) (50 and 75 mg/kg) Against HCT 116 Bearing SCID Mice

The median tumor burden at start of therapy was 198 to 221 mm³. As single agents, Compound (1) (5 mg/kg/administration (Adm)), Compound (2b) (30 mg/kg/adm) and Compound (2a) (50 and 75 mg/kg/adm) were administered PO daily from days 11 to 18 post tumor implantation. In the combination groups, the dose of Compound (1) was combined with each dose of Compound (2a) and Compound (2b), as shown in Table 2.

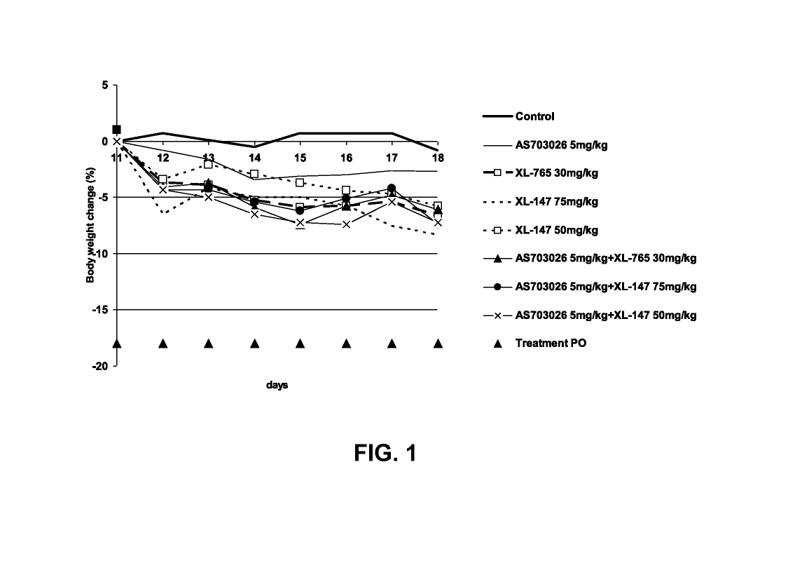

As single agents or used in combination, Compound (1) and Compound (2a) were well-tolerated, inducing minimal BWL (FIG. 1 and Table 2). As single agents, Compound (1), Compound (2a) and Compound (2b) achieved a ΔT/ΔC>40%) under these test conditions.

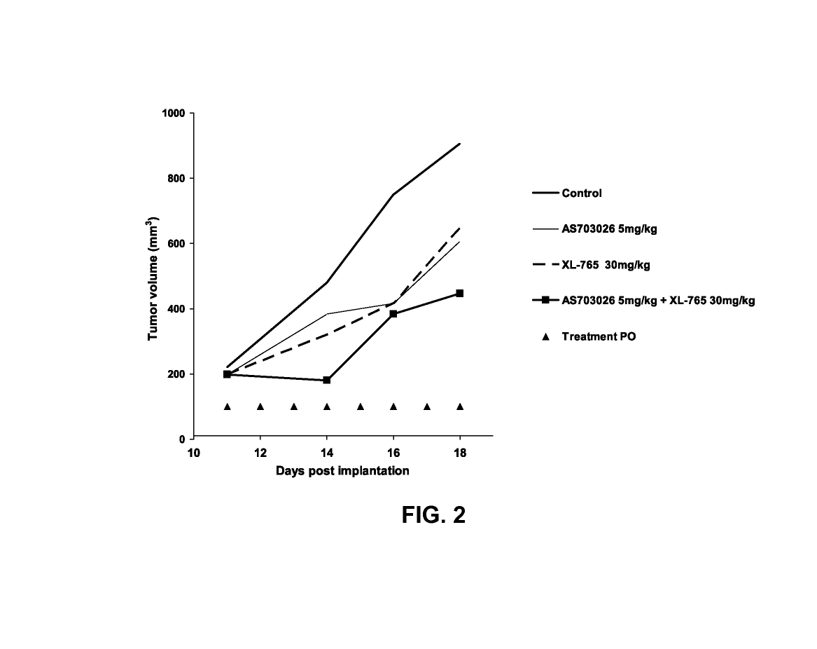

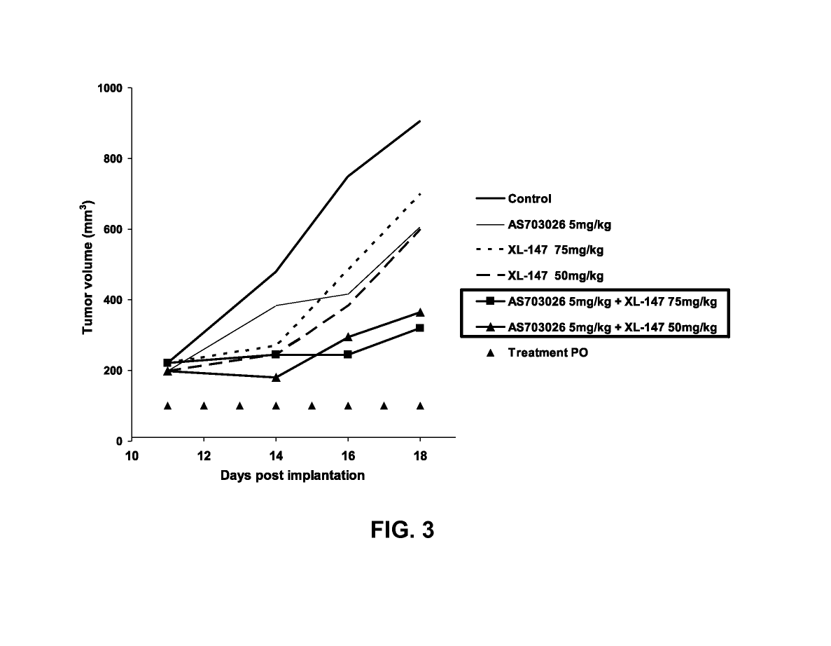

In combination, treatment with Compound (1) at 5 mg/kg/adm and Compound (2b) at 30 mg/kg/adm achieved a ΔT/ΔC of 27% (FIG. 2 and Table 1), but as shown by Table 3, therapeutic synergy was not reached (p=0.0606 for global analysis). Treatment with Compound (1) at 5 mg/kg/adm and Compound (2a) at 50 and 75 mg/kg/adm achieved a ΔT/ΔC of 22% and 21%, respectively (FIG. 3 and Table 2). As shown by Table 2, therapeutic synergy was achieved for both combinations (p=0.0091 and p<0.0001 globally, respectively). See also Tables 11A and 11B.

Second Study: Antitumor Activity of Compound (1) (10 and 20 mg/kg) in Combination with Compound (2b) (20 mg/kg) and Compound (1) (10 mg/kg) in Combination With Compound (2a) (50 and 75 mg/kg) Against HCT 116 Bearing SCID Mice

The median tumor burden at start of therapy was 180 to 198 mm³. As single agents, Compound (1) (10 and 20 mg/kg/adm), Compound (2b) (20 mg/kg/adm) and Compound (2a) (50 and 75 mg/kg/adm) were administered PO daily from days 11 to 18 post tumor implantation. In the combination groups, the dose of Compound (1) was combined with each dose of Compound (2a) and Compound (2b), as shown in Table 3.

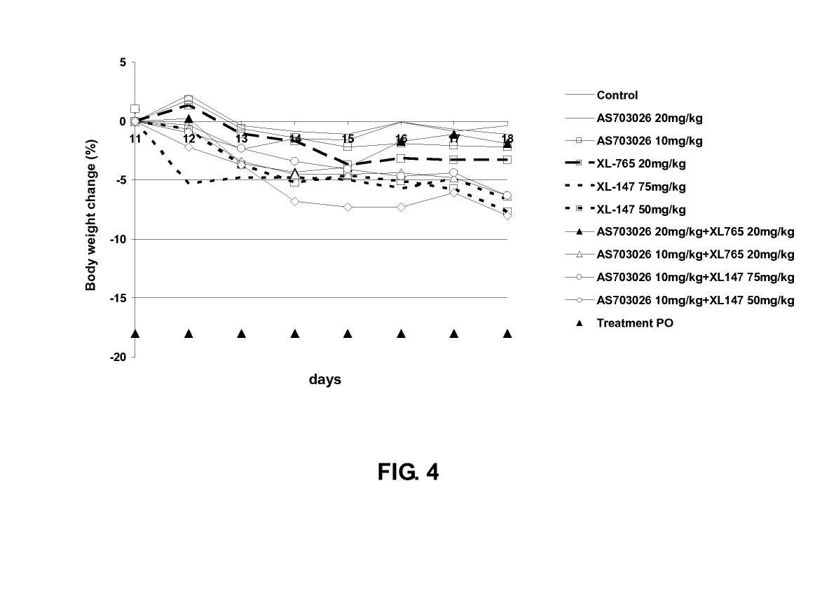

As single agents, Compound (1), Compound (2a) and Compound (2b) were well-tolerated, inducing minimal BWL (FIG. 4 and Table 4).

As single agents, Compound (1) (10 and 20 mg/kg/adm) achieved a ΔT/ΔC of 20% and 22%, respectively, while Compound (2b) at 20 mg/kg/adm achieved a ΔT/ΔC>40%. As shown in Table 4, Compound (2a) at both doses tested achieved a ΔT/ΔC>40%.

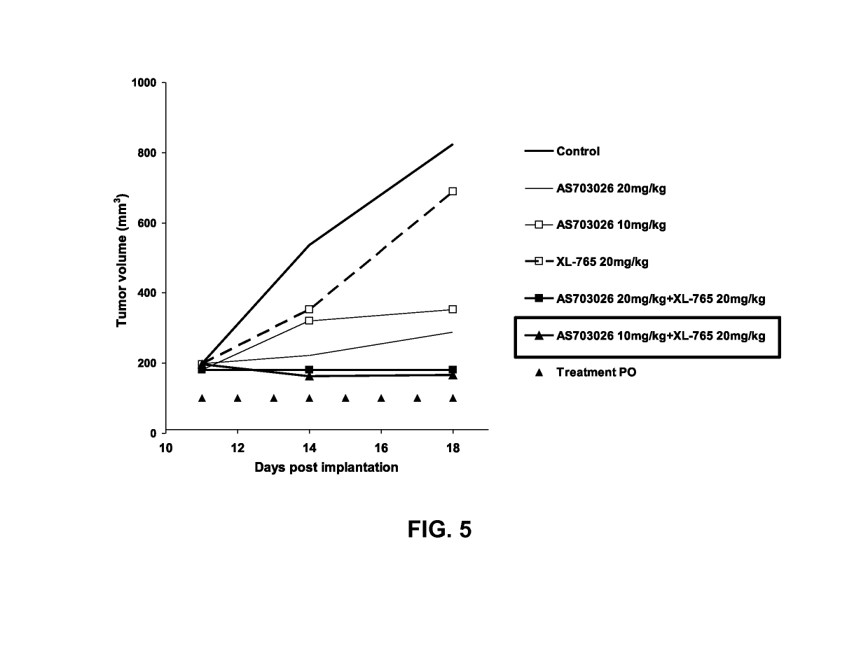

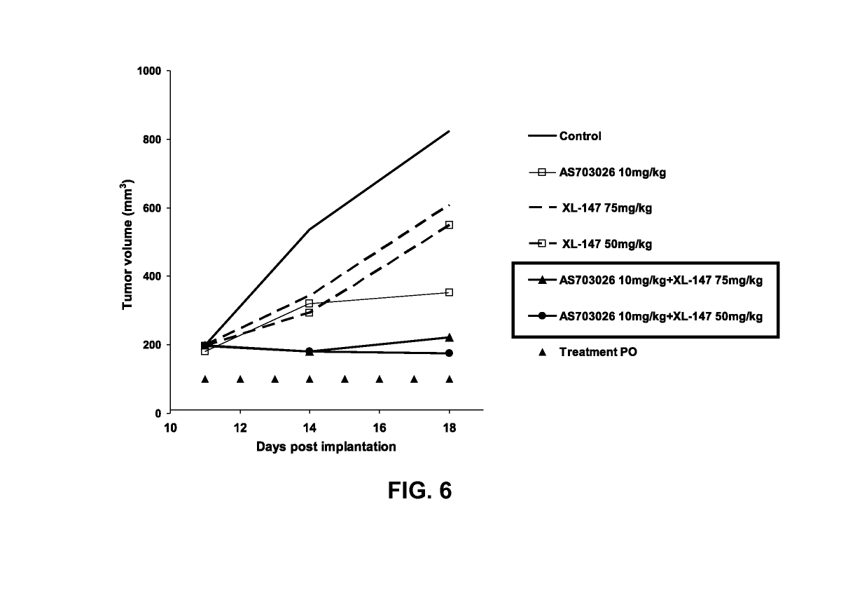

In combination, treatment with Compound (1) at 10 or 20 mg/kg/adm and Compound (2b) at 20 mg/kg/adm achieved a ΔT/ΔC of 0, and therapeutic synergy was reached with Compound (1) at 10 mg/kg/adm (p=0.0004 globally). As shown by Table 5, therapeutic synergy was not reached with Compound (1) at 20 mg/kg/adm (p=0.2169 globally). Partial regression (PR) was observed in 2/7 mice for the combination treatment of Compound (1) at 10 mg/kg/adm and Compound (2b) at 20 mg/kg/adm (FIG. 5 and Table 4). When Compound (1) was used at 10 mg/kg/adm, the combinations with Compound (2a) at 75 and 50 mg/kg/adm achieved, respectively a ΔT/ΔC of 5% and ΔT/ΔC<0, with 1/7 PR occurring for both combination treatments (FIG. 6 and Table 4). As shown by Table 5, both combinations (p=0.0063 and p=0.0019 globally, respectively) achieved therapeutic synergy. In all combination groups, tumor stasis was achieved (FIG. 5 and FIG. 6). See also Tables 12A and 12B below.

Third Study: Antitumor Activity of Compound (1) (10 and 20 mg/kg) in Combination With Compound (2a) (50 and 75 mg/kg) Against HCT 116 Bearing SCID Mice

The median tumor burden at start of therapy was 187 to 189 mm³. As single agents, Compound (1) (10 and 20 mg/kg/adm) and Compound (2a) (50 and 75 mg/kg/adm) were administered PO daily from days 11 to 20 post tumor implantation. In the combination groups, the dose of Compound (1) was combined with each dose of Compound (2a), as shown in Table 6.

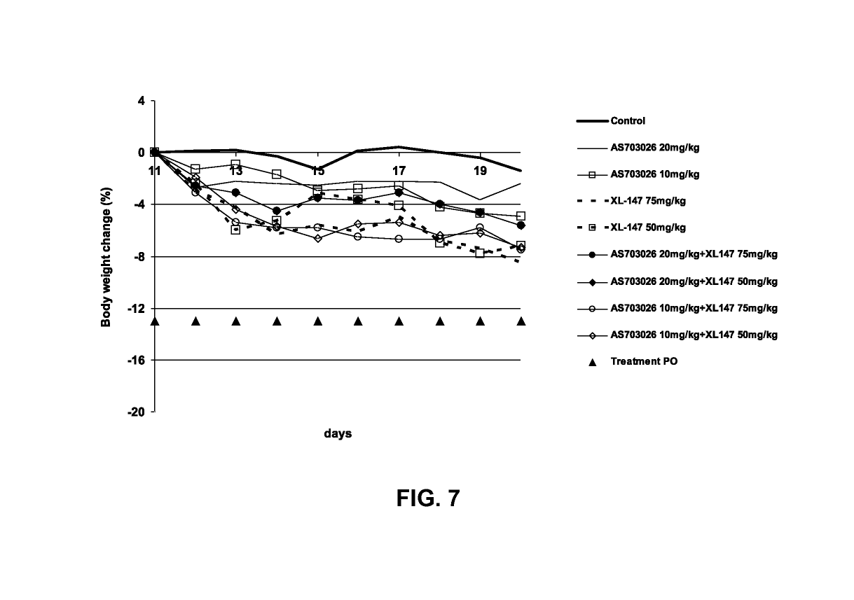

As single agents, Compound (1) and Compound (2a) were well-tolerated, inducing minimal BWL (FIG. 7 and Table 6).

As a single agent, Compound (1) achieved a ΔT/ΔC of 34% at a dose of 20 mg/kg/adm and ΔT/ΔC>40% at a dose of 10 mg/kg/adm (FIG. 7). As shown in Table 6, Compound (2a) at both doses tested achieved a ΔT/ΔC>40%.

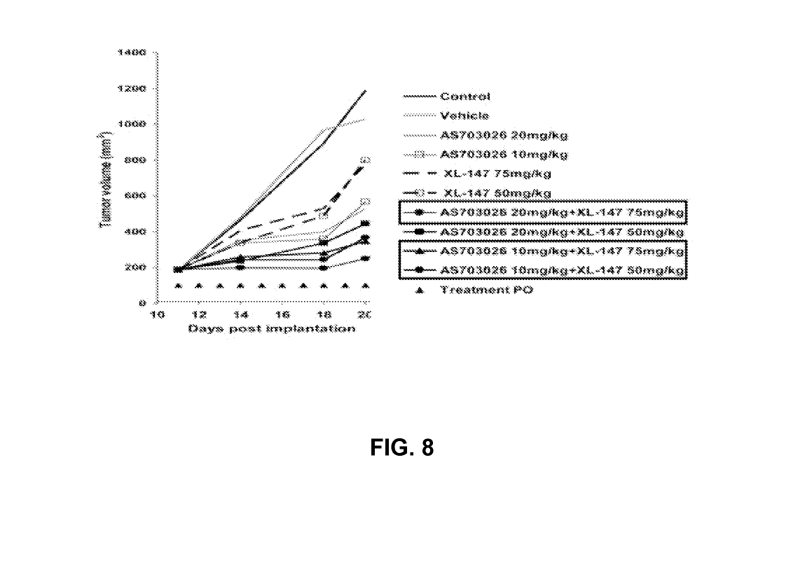

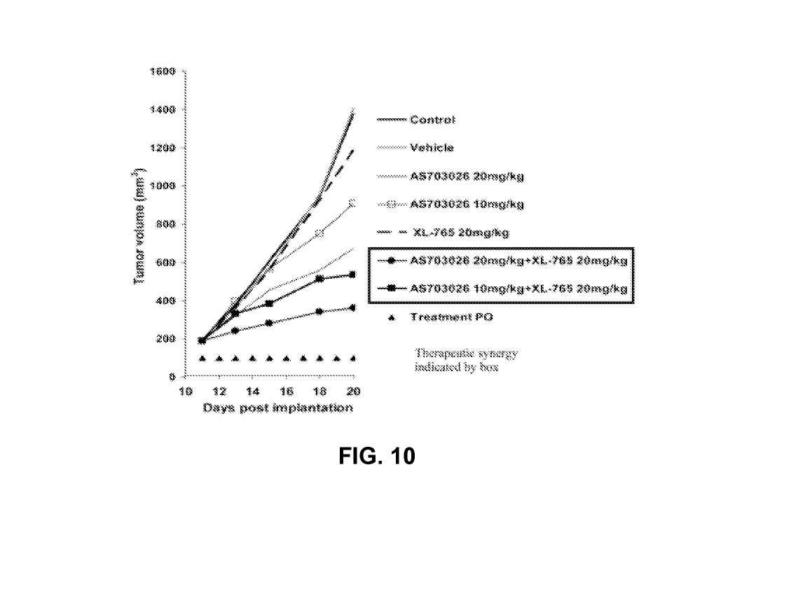

In the combination, treatment with Compound (1) at 10 or 20 mg/kg/adm and Compound (2a) at 75 mg/kg/adm achieved ΔT/ΔC of 18% and 9%, respectively) (FIG. 10 and Table 6), and therapeutic synergy was reached (p=0.0109 and p=0.0003 globally, respectively) (Table 6). The treatment with Compound (1) at 10 or 20 mg/kg/adm and Compound (2a) at 50 mg/kg/adm achieved ΔT/ΔC of 19% and 22%, respectively) (FIG. 10 and Table 6). Therapeutic synergy was reached only for the combination with Compound (1) at 10 mg/kg (p=0.0088 globally) (Table 7). As shown by Table 7, therapeutic synergy was not reached with Compound (1) at 20 mg/kg/adm (p=0.0764 globally). In all combination groups, tumor stasis was achieved (FIG. 8). See also Table 13 below.

Fourth Study: Antitumor Activity of Compound (1) (10 and 20 mg/kg) in Combination With Compound (2b) (20 mg/kg) Against HCT 116 Bearing SCID Mice

The median tumor burden at start of therapy was 189 to 196 mm³. As single agents, Compound (1) (10 and 20 mg/kg/adm) and Compound (2b) (20 mg/kg/adm) were administered PO daily from days 11 to 20 post tumor implantation. In the combination groups, the dose of Compound (2b) was combined with each dose of Compound (1), as shown in Table 8.

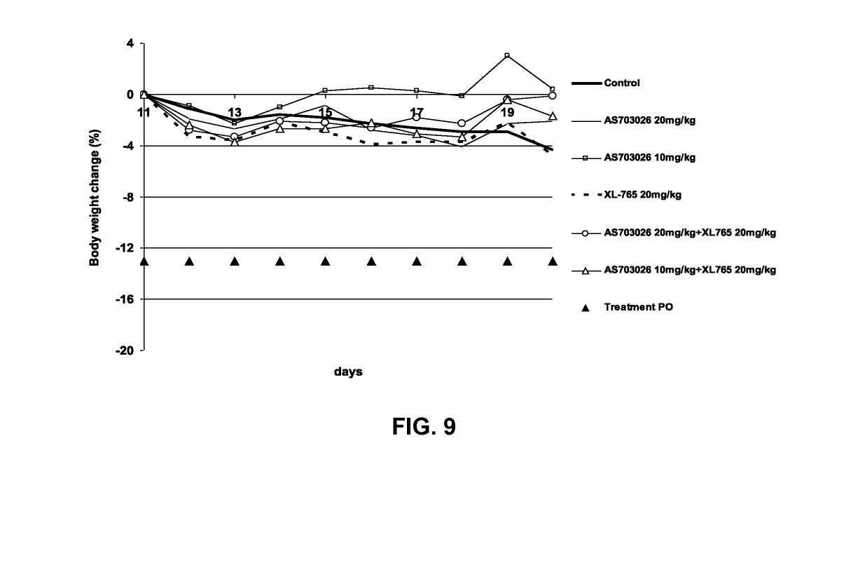

As single agents, Compound (1) and Compound (2b) were well-tolerated, inducing minimal BWL (FIG. 9 and Table 8).

As single agents, Compound (1) (10 and 20 mg/kg/adm) and Compound (2b) at 20 mg/kg achieved a ΔT/ΔC>40% (FIG. 10 and Table 8).

In the combination, the treatment with Compound (1) at 10 or 20 mg/kg/adm and Compound (2b) at 20 mg/kg/adm achieved a ΔT/ΔC of 30% and 15%, respectively (FIG. 10 and Table 8), and therapeutic synergy was reached (p=0.0002 and p=0.0008 globally, respectively) (Table 9). See also Table 14 below.

### Example 3

### In Vivo Activity of Compound (1) in Combination with Compound (2a) or Compound (2b) Against Subcutaneous Human Pancreatic MiaPaCa-2 Bearing Nude Mice

To evaluate the antitumor activity of the MEK inhibitor Compound (1) (5 mg/kg) in combination with the pan-PI3K inhibitor Compound (2a) (50 mg/kg) or the dual pan-PI3K/mTOR inhibitor Compound (2b) (30 mg/kg), experiments were conducted using female nude mice bearing human pancreatic MiaPaCa-2 (KRAS mutant) xenografts.

A low dose of Compound (1) at 5 mg/kg was tested in combination with Compound (2b) at 30 mg/kg and Compound (2a) at 50 mg/kg.

### Materials and Methods

The human pancreatic cancer cell line MiaPaCa-2 (American Type Culture Collection, Manassas Va.), was cultured in MEM medium containing 10% fetal bovine serum, 1% essential amino acid, 1% sodium pyruvate (Life Technologies, Carlsbad, Calif.). Cells were trypsonized during the log phase of growth at 60-85% confluence, collected and washed once with PBS. Cells were re-suspended in PBS (Life Technologies, Carlsbad, Calif.) and then mixed 1:1 with Matrigel (BD Biosciences, San Jose, Calif.). Cells were stored at 4° C. until implantation.

MiaPaCa-2 cells (10×10⁶ in a 200 μl PBS:Matrigel (1:1) suspension) were subcutaneously injected into the right flank area of female nude (Crl:NU-Foxn1nu) mice (6-8 weeks old, Charles River Laboratories, Wilmington, Mass.). All mice in this study were used according to the guidelines approved by the EMD-Serono Institutional Care and Animal Use Committee (IACUC), #07-003.

A solution of 0.5% CMC (carboxymethylcellulose; Sigma-Aldrich, St. Louis, Mo.) and 0.25% Tween 20 (Acros Organics, Morris Plains, N.J.) in water was used as the vehicle for this study. Compound (1) (Lot #27) was prepared by suspending 10 mg of compound in 20 mL of 0.5% CMC 0.25% Tween 20 in water to make a 0.5 mg/mL (5.0 mg/kg) dosing solution.

Compound (2a) was weighed (5 mg for 1 mL of solution) and water added for injection (60% of final volume i.e. 0.60 ml). Solution was mixed via five cycles of vortexing and sonicating in a sonicating water bath for 1 min each. Completed with water for dosing. Compound (2b) was weighed (3 mg for 1 mL of solution), 10 μL HCl 1N was added and then water was added for injection (60% of final volume i.e. 0.60 ml). Solution was mixed via five cycles of vortexing and sonicating in a sonicating water bath for 1 min each. 1N NaOH was added to adjust the pH up to 3 and finally completed with water for injection.

Developing tumors located in the right flank area of female nude mice were measured over time with digital calipers. Seven days after cell implantation, the tumors had reached an average volume of 165 mm³ in an ample number of mice to begin the study. Mice bearing a tumor that was significantly different from the average tumor volume were excluded from the study. The remaining tumor-bearing mice were randomized into seven experimental groups (n=9), so that each group had the same mean tumor volume.

In all combination groups, both agents were administered to the animals at the same time, within approximately 5-10 minutes of each other. The treatments began on the seventh day following implantation of the Miapaca-2 cells, which was designated as Day 0 for data evaluation purposes. Animals underwent 21 days of treatment. Body weights and tumor volumes were assessed twice per week post treatment initiation. On Day 22, all animals were euthanized via progressive hypoxia with CO₂.

Efficacy was determined by analyzing tumor volumes and the percent ΔT/ΔC (% ΔT/ΔC). Tumor volume was determined by using the tumor length (1) and width (w) measurements and calculating the volume with the equation 1*w²/2. The length was measured along the longest axis of the tumor and width was measured perpendicular to that length. The mean percent of actual tumor growth inhibited by the treatments was calculated as follows: [% ΔT/ΔC=((TV_{f}−TVᵢTV_{fctr}−TV_{iCtrl}))×100%], where TV=tumor volume, f=final, i=initial and Ctrl=control group. Tolerability was assessed by regarding percent body weight difference during the treatment period. Percent body weight difference was calculated as follows: [% Body weight difference=(BW_{c}−BWᵢ)/BWᵢ×100%], where BW=body weight, c=current, i=initial.

Tumor volume data and percent body weight differences were analyzed by Repeated Measures Analysis of Variance (RM-ANOVA) followed by Tukey's post-hoc multiple pairwise comparisons (α=0.05).

### Results of In Vivo Studies

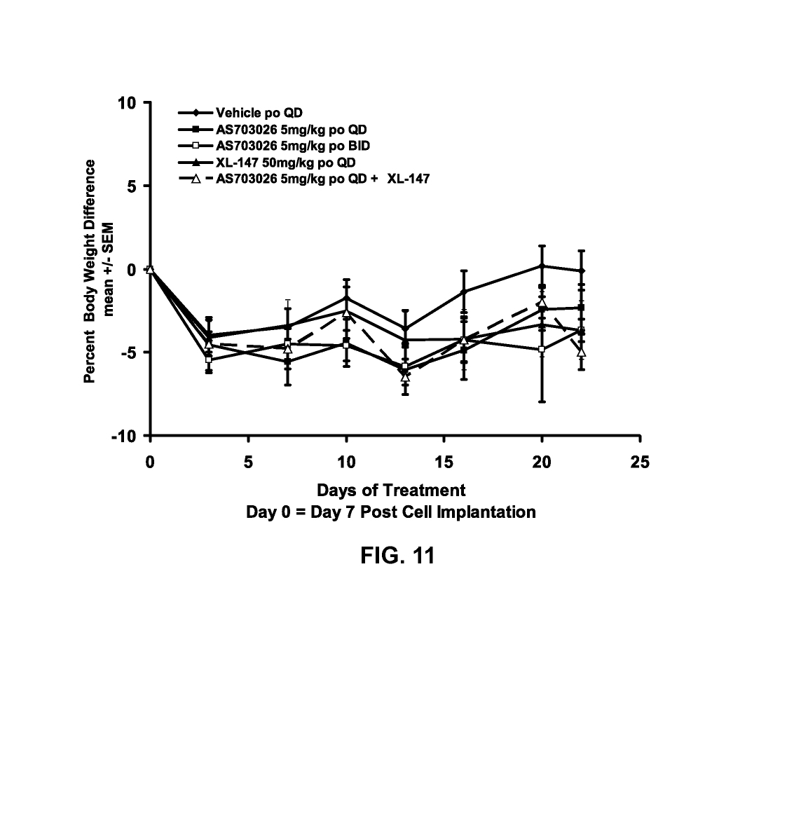

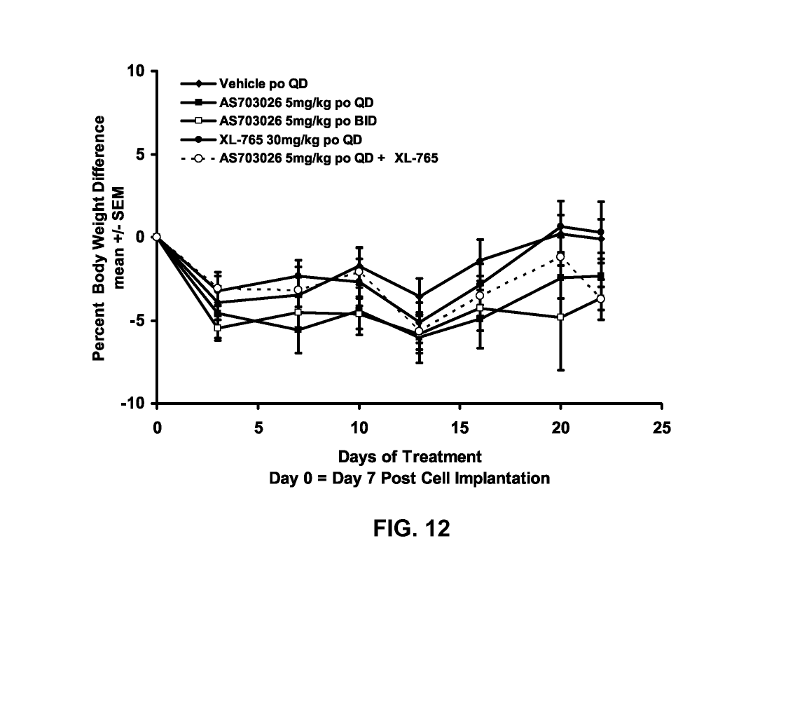

No groups experienced more than 5% body weight loss during the study. No clinical signs were noted (FIG. 11) for the combination with Compound (2a) or (FIG. 12) for the combination with Compound (2b).

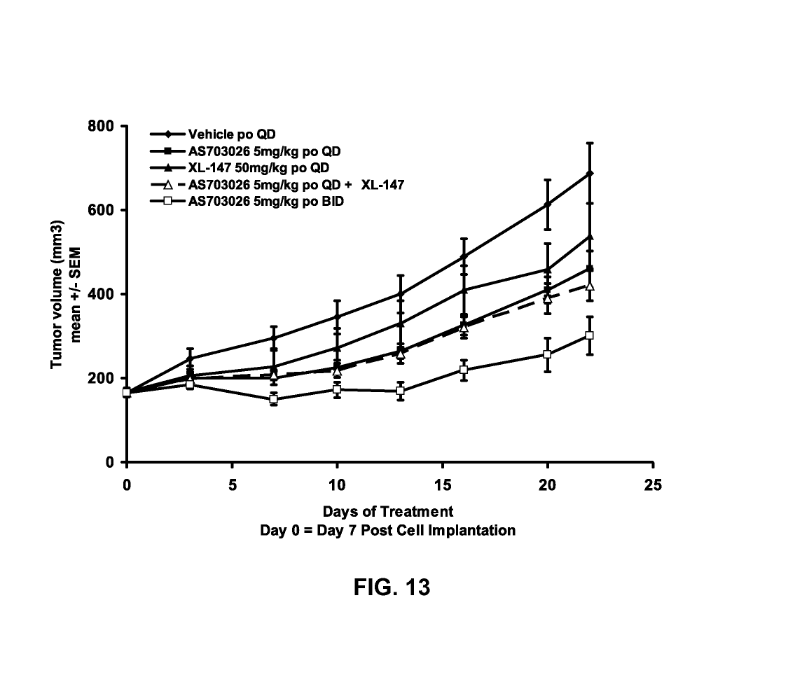

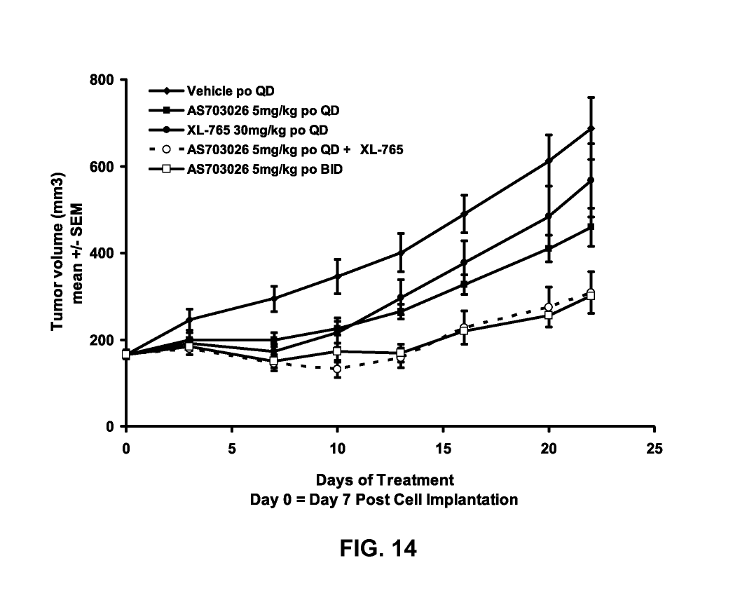

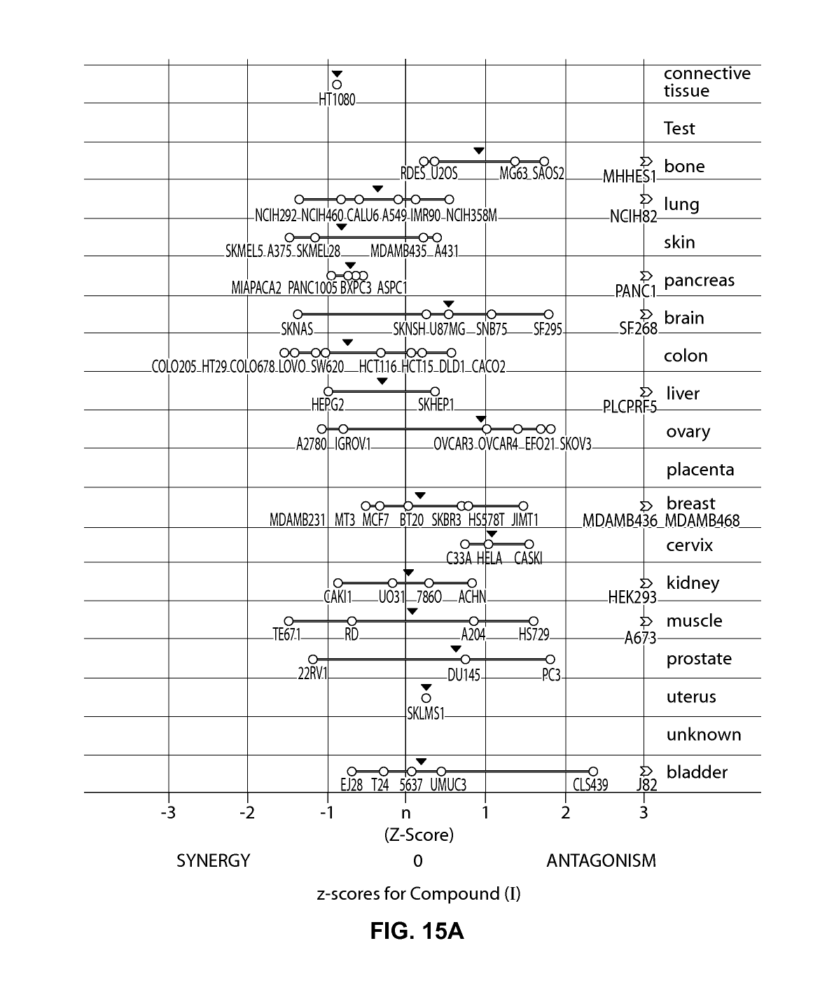

As single agents, Compound (1) (5 mg/kg/adm), Compound (2a) (50 mg/kg) and Compound (2b) (30 mg/kg) achieved ΔT/ΔC>40% in these assays (FIGS. 13 and 14 and Table 10).

In combination, treatment with Compound (1) at 5 mg/kg/adm and Compound (2b) at 30 mg/kg/adm achieved ΔT/ΔC=27.3% (FIG. 14 and Table 10), and therapeutic synergy was reached (p<0.05) (Table 10). In contrast, the treatment with Compound (1) at 5 mg/kg/adm and Compound (2a) at 50 mg/kg/adm achieved ΔT/ΔC>40% (FIG. 13 and Table 10), and therapeutic synergy was not reached (p>0.05) (Table 10).

### Summary of In Vivo Results

The in vivo work presented here reports the in vivo antitumor activity of combining Compound (1), an oral potent and selective allosteric inhibitor of MEK1/2, with oral, potent, and specific inhibitors of class I PI3K lipid kinases Compound (2a), a pan-PI3K inhibitor, and Compound (2b), a dual pan-PI3K and mTOR inhibitor. This work has been performed against human colon carcinoma HCT 116 xenografts harboring a G13D activating mutation of KRAS and an activating mutation of PIKC3A known to reduce the sensitivity to MEK inhibition and against human pancreatic MiaPaCa-2 xenografts harboring a KRAS mutation.

In the studies described above, combination treatment was highly effective in inducing a sustained tumor stasis during the treatment phase and realizing therapeutic synergy.

In conclusion, a potent antitumor activity with therapeutic synergy has been achieved in PIKC3A and KRAS mutant HCT 116 driven xenograft model when combining the inhibitor of MEK1/2 Compound (1) with Compound (2a), a pan-PI3K inhibitor, and in both PIKC3A and KRAS mutant HCT 116 driven xenograft model and KRAS mutant MiaPaCa-2 driven xenograft model, when combining Compound (1) with Compound (2b), a dual pan-PI3K and mTOR inhibitor.

### Example 4

### Fluorescence Molecular Tomography Study of Combination of Compound (1) with Compound (2b) or Compound (2b) Against Subcutaneous Human Colon Carcinoma HCT 116 Bearing SCID Mice

To evaluate the apoptotic activity of the MEK inhibitor Compound (1) in combination with the pan-PI3K inhibitor Compound (2a) or the dual pan-PI3K/mTOR inhibitor Compound (2b), experiments were conducted using female SCID mice bearing human colon carcinoma HCT 116 (KRAS and PIK3CA mutant) xenografts in which apoptosis induction was monitored non-invasively using fluoresence molecular tomography (FMT).

### Methods

HCT116 tumor cells were implanted subcutaneously in the intra-scapular region in SCID mice. Implanted animals received 50 mg/kg Compound (2a) or 20 mg/kg Compound (2b) from day 11 to day 17, as single agents or combined with 10 mg/kg Compound (1). Each agent was given by oral route on a daily schedule. Tumor growth was monitored throughout the experiment by callipering the tumors. To quantify apoptosis, fluorescent Annexin-Vivo-750 was injected intravenously one hour post daily treatment on days three and seven after start of treatment. Animals were imaged by FMT three hours post probe injection to document fluorescent Annexin uptake in the tumor. Ex vivo apoptosis was assessed on tumor lysates using Meso Scale Discovery assays for cleaved caspase-3 and cleaved-PARP detection.

### Results

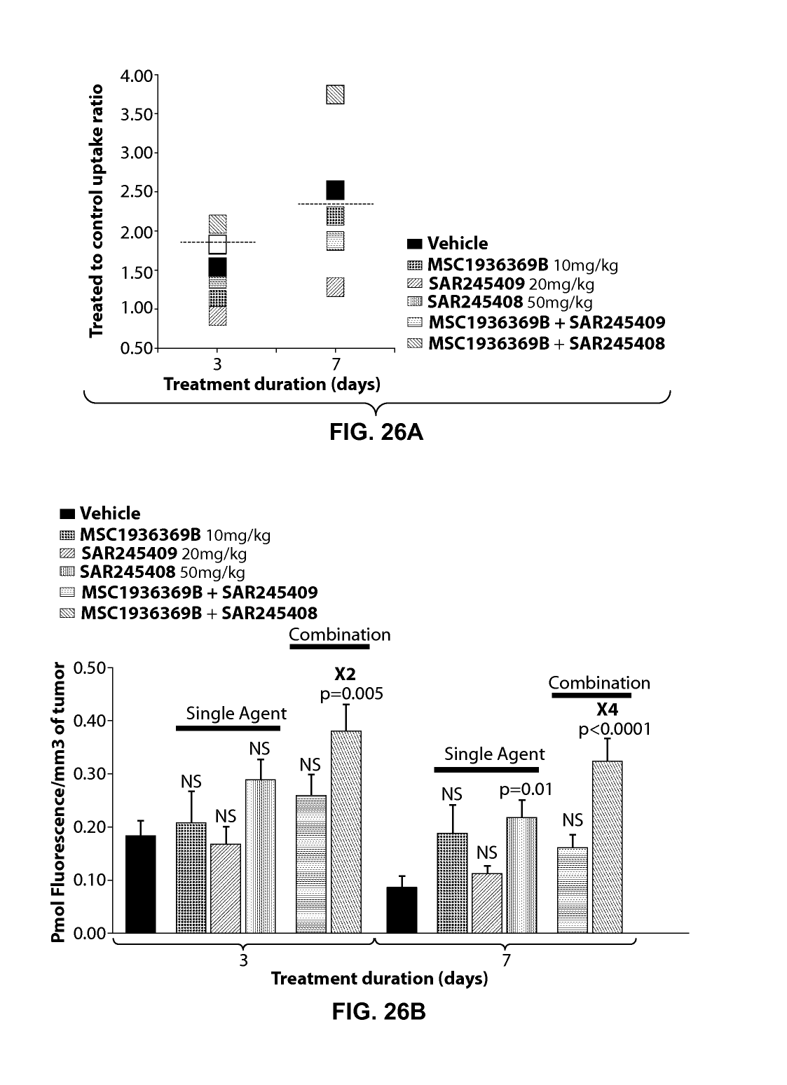

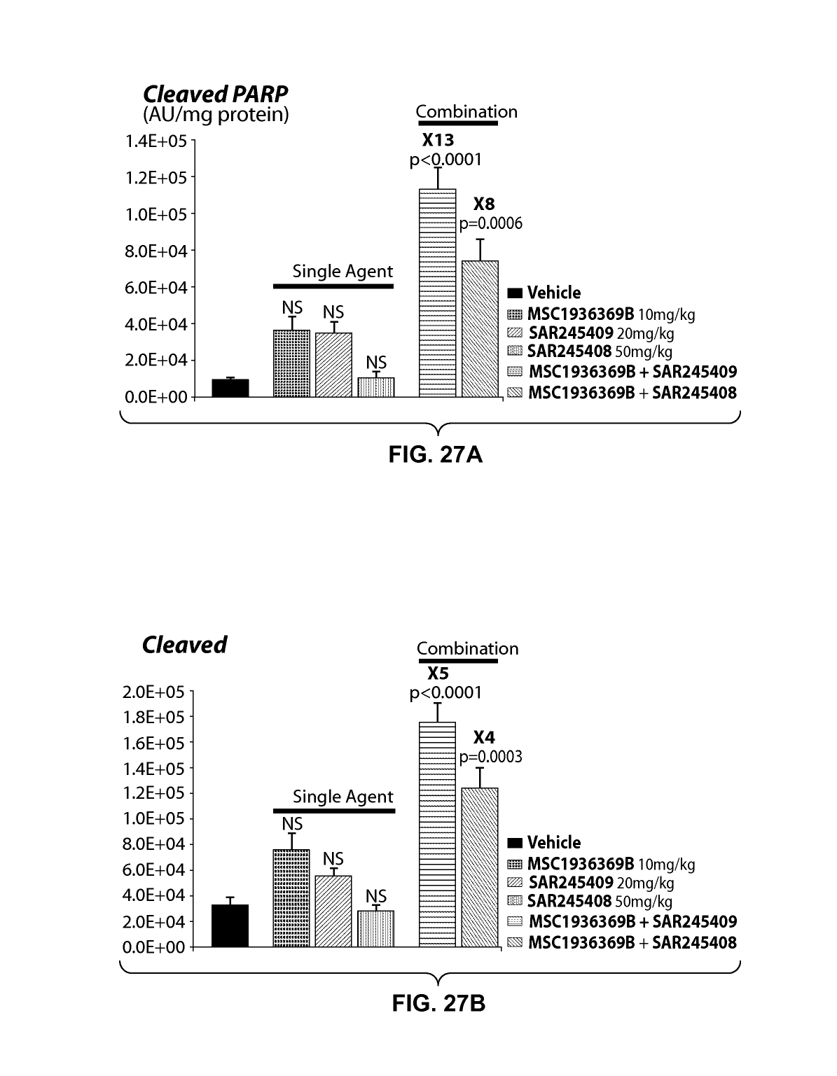

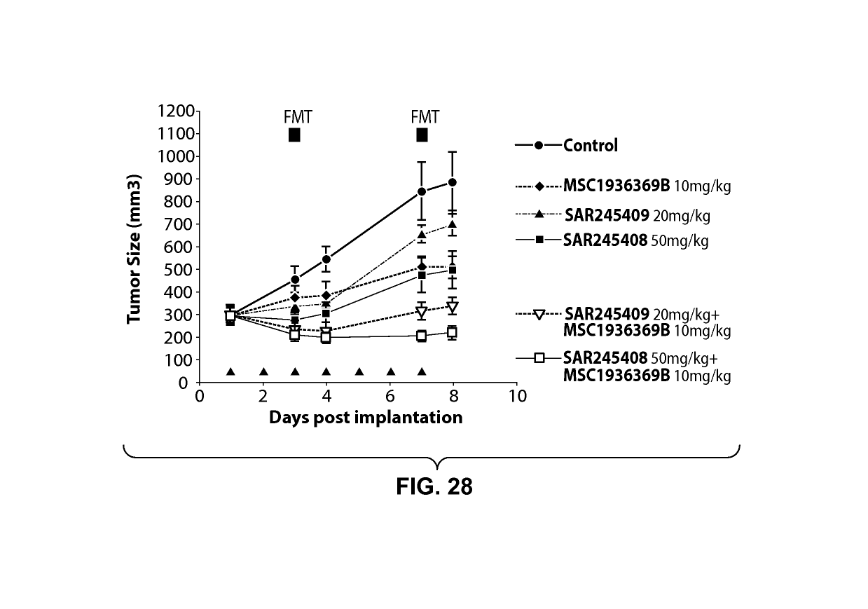

Under these regimens, Compound (1), Compound (2a) and Compound (2b) used as single agents showed marginal activity on HCT116 tumor growth with ΔT/ΔC=40% (NS), 36% (p=0.023) and 80% (NS) respectively at the end of study (FIG. 28). Conversely, both Compound (2a) and Compound (2b) in combination with Compound (1) induced strong tumor growth inhibition (ΔT/ΔC<0, associated with 23% median tumor regression (p<0.0001) for Compound (2a)/Compound (1) and (ΔT/ΔC<0 with 5% median tumor regression (p=0.0009) for Compound (2b)/Compound (1)). Both combination therapies were associated with a clear enhancement of ex vivo cleaved caspase-3 (3.7 & 5.2 fold) (FIG. 27B) and cleaved-PARP (8.4 & 12.8 fold) (FIG. 27A) after four days treatment. Compound (2a)/Compound (1) combination therapy was associated with a significant enhancement of Annexin-V-750 uptake in the tumor, reflecting apoptosis induction after three and seven days of combined therapy (p=0.005 and <0.0001) (FIG. 26B). The ratios of Annexin fluorescence in treated animal groups relative to control were respectively 2.1 after 3 days and 3.8 after 7 days of combination therapy (FIG. 26A).

### SUMMARY

The combination of the MEK1/2 inhibitor Compound (1) with the Pan-PI3K inhibitor Compound (2a) or the Pan-PI3K/mTOR Compound (2b) resulted in significantly enhanced anti-tumor activity in a dual KRAS/PIK3CA mutated tumor xenograft model, with synergistic induction of tumor apoptosis as demonstrated ex vivo for both combinations and in vivo using longitudinal FMT imaging for the Compound (2a)/Compound (1) combination.

### Example 5

### In Vivo Activity of Compound (1) in Combination with Compound (2b) or Compound (2a) Against Subcutaneous Human Colon Tumors CR-LRB-009C Bearing SCID Female Mice

To evaluate the antitumor activity of the MEK inhibitor Compound (1) in combination with the pan-PI3K inhibitor Compound (2a) or the dual pan-PI3K/mTOR inhibitor Compound (2b), experiments were conducted using female SCID mice bearing human primary colon tumors CR-LRB-009C (KRAS and PIK3CA mutant) xenografts. In this study, Compound (1) at 20 mg/kg was tested in combination with Compound (2b) at 20 mg/kg and Compound (2a) at 75 mg/kg.

### Materials And Methods

CB17/1CR-Prkdc severe combined immunodeficiency (SCID)/Crl mice, at 8-10 weeks old, were bred at Charles River France (Domaine des Oncins, 69210 L'Arbresle, France) from strains obtained from Charles River, USA. Mice were over 18 g at start of treatment after an acclimatization time of at least 5 days. The mice had free access to food (UAR reference 113, Villemoisson, 91160 Epinay sur Orge, France) and sterile water. The mice were housed on a 12 hours light/dark cycle. Environmental conditions including animal maintenance, room temperature (22° C.±2° C.), relative humidity (55%±15%) and lighting times were recorded by the supervisor of laboratory animal sciences and welfare (LASW) and archived.

The human primary colon carcinoma CR-LRB-009C tumor model was established by implanting (SC) small tumor fragments and was maintained in SCID female mice using serial passages.

Compound (1) formulation was prepared by incorporating the MEK inhibitor into 0.5% CMC 0.25% Tween 20. The preparation was stored at 4° C. and resuspended by vortexing before use. The oral form of the compound was prepared every 3 days. The volume of administration per mouse was 10 mL/kg.

Compound (2a) formulation was prepared in water for injection. The stock solution was chemically stable 7 days in the dark at 4° C. The volume of administration per mouse was 10 mL/kg.

Compound (2a) and Compound (2b) formulations were prepared in 1N HCl and water for injection, final pH was 3, followed by five cycles of vortexing and sonicating. The stock solution was chemically stable 7 days in the dark at 4° C. The volume of PO administration per mouse was 10 mL/kg.

For subcutaneous implantation of tumor cells, skin in the flank of the mice was disinfected using alcohol or Betadine® solution (Alcyon) and a suspension of tumor cells was inoculated SC unilaterally under a volume of 0.2 mL using a 23 G needle.

The dosages and schedule of administration of Compound (1), Compound (2a) and Compound (2b) used as single agent or in combination are described in the results section and detailed in Tables 15-17.

The animals required to begin a given experiment were pooled and implanted monolaterally on day 0. Treatments were administered on measurable tumors. The solid tumors were allowed to grow to the desired volume range (animals with tumors not in the desired range were excluded). The mice were then pooled and unselectively distributed to the various treatment and control groups. Treatment started 11 days post CR-LRB-009C tumor fragment implantation as indicated in the results section and in each table. The dosages are expressed in mg/kg, based on the body weight at start of therapy. Mice were checked daily, and adverse clinical reactions noted. Each group of mice was weighed as a whole daily until the weight nadir was reached. Then, groups were weighed once to thrice weekly until the end of the experiment. Tumors were measured with a caliper 2 to 3 times weekly until final sacrifice for sampling time, tumor reached 2000 mm³ or until the animal died (whichever comes first). Solid tumor volumes were estimated from two-dimensional tumor measurements and calculated according to the following equation:

Tumor weight(mg)=Length(mm)×Width²(mm²)/2

The day of death was recorded. Surviving animals were sacrificed and macroscopic examination of the thoracic and abdominal cavities was performed.

A dosage producing a 15% body weight loss (BWL) during three consecutive days (mean of group), 20% BWL during 1 day or 10% or more drug deaths was considered an excessively toxic dosage. Animal body weights included the tumor weight.

The primary efficacy end points are ΔT/ΔC, percent median regression, partial and complete regressions (PR and CR). Statistical analyses were performed on SAS system release 8.2 for SUN4 via Everstat V5 software and SAS 9.2 software. A probability less than 5% (p<0.05) was considered as significant.

### Results of In Vivo Studies

The median tumor burden at start of therapy was 126 to 144 mm³. As single agents, Compound (1) (20 mg/kg/administration (Adm)), Compound (2b) (20 mg/kg/adm) and Compound (2a) (75 mg/kg/adm) were administered PO daily from days 11 to 21 post tumor implantation. In the combination groups, the dose of Compound (1) was combined with each dose of Compound (2a) and Compound (2b), as shown in Table 15.

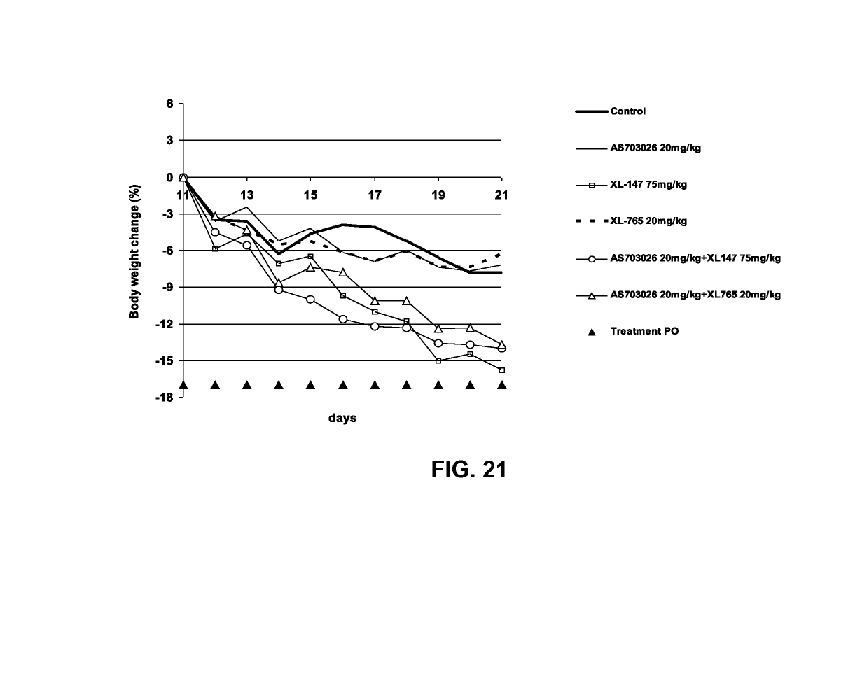

As single agents or used in combination, Compound (1), Compound (2b) and Compound (2a) were tolerated, inducing some BWL but not reaching toxicity (FIG. 21 and Table 15). As single agents, Compound (1) and Compound (2b) achieved a ΔT/ΔC>40%, while Compound (2a) achieved a ΔT/ΔC of 39% under these test conditions.

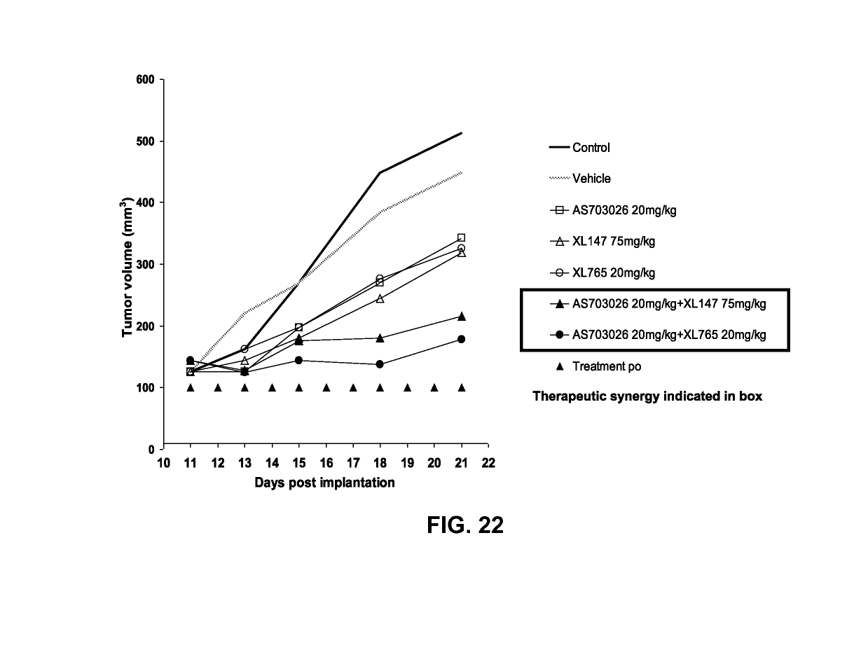

In the combination, the treatment with Compound (1) at 20 mg/kg/adm and Compound (2b) at 20 mg/kg/adm achieved a ΔT/ΔC of 4% (FIG. 22 and Table 15), and as shown by Table 16, therapeutic synergy was reached (p<0.0001 for global analysis). The treatment with Compound (1) at 20 mg/kg/adm and Compound (2a) at 75 mg/kg/adm achieved a ΔT/ΔC of 21% (FIG. 22 and Table 15), and as shown by Table 16, therapeutic synergy was achieved (p=0.0386 globally). See also Table 17.

### Summary of In Vivo Results

The in vivo work presented here reports the in vivo antitumor activity of combining Compound (1), an oral potent and selective allosteric inhibitor of MEK1/2, with oral, potent, and specific inhibitors of class I PI3K lipid kinases Compound (2a), a pan-PI3K inhibitor, and Compound (2b), a dual pan-PI3K and mTOR inhibitor. This work has been performed against human primary colon carcinoma CR-LRB-009C xenografts harboring a dual KRAS and PIKC3A mutation known to reduce the sensitivity to MEK inhibition.

In the study, combination treatment induced a sustained tumor stasis during the treatment phase and reached therapeutic synergy.

Accordingly, a potent antitumor activity with therapeutic synergy has been achieved in a PIKC3A- and KRAS-mutant CR-LRB-009C driven xenograft model when combining the inhibitor of MEK1/2 Compound (1) with Compound (2a), a pan-PI3K inhibitor or Compound (2b), a dual pan-PI3K and mTOR inhibitor.

### Example 6

### In Vivo Activity of Compound (1) in Combination with Compound (2a) or Compound (2b) Against Subcutaneous Human Colon Tumors CR-LRB-013P Bearing SCID Female Mice

To evaluate the antitumor activity of the MEK inhibitor Compound (1) in combination with the pan-PI3K inhibitor Compound (2a) or the dual pan-PI3K/mTOR inhibitor Compound (2b), experiments were conducted using female SCID mice bearing human primary colon tumors CR-LRB-013P (KRAS mutant) xenografts. In this study, Compound (1) at 20 mg/kg was tested in combination with Compound (2b) at 20 mg/kg or Compound (2a) at 75 mg/kg.

### Materials and Methods

CB17/1CR-Prkdc severe combined immunodeficiency (SCID)/Crl mice, at 8-10 weeks old, were bred at Charles River France (Domaine des Oncins, 69210 L'Arbresle, France) from strains obtained from Charles River, USA. Mice were over 18 g at start of treatment after an acclimatization time of at least 5 days. The mice had free access to food (UAR reference 113, Villemoisson, 91160 Epinay sur Orge, France) and sterile water. The mice were housed on a 12 hours light/dark cycle. Environmental conditions including animal maintenance, room temperature (22° C.±2° C.), relative humidity (55%±15%) and lighting times were recorded by the supervisor of laboratory animal sciences and welfare (LASW) and archived.

The human primary colon carcinoma CR-LRB-013P tumor model was established by implanting (SC) small tumor fragments and was maintained in SCID female mice using serial passages.

Compound (1) formulation was prepared by incorporating the MEK inhibitor into 0.5% CMC 0.25% Tween 20. The preparation was stored at 4° C. and resuspended by vortexing before use. The oral form of the compound was prepared every 3 days. The volume of administration per mouse was 10 mL/kg.

Compound (2a) formulation was prepared in water for injection. The stock solution was chemically stable 7 days in the dark at 4° C. The volume of administration per mouse was 10 mL/kg.

Compound (2a) and Compound (2b) formulations were prepared in 1N HCl and water for injection, final pH was 3, followed by five cycles of vortexing and sonicating. The stock solution was chemically stable 7 days in the dark at 4° C. The volume of PO administration per mouse was 10 mL/kg.

For subcutaneous implantation of tumor cells, skin in the flank of the mice was disinfected using alcohol or Betadine® solution (Alcyon) and a suspension of tumor cells was inoculated SC unilaterally under a volume of 0.2 mL using a 23 G needle.

The dosages and schedule of administration of Compound (1), Compound (2a) and Compound (2b) used as single agent or in combination are described in the results section and detailed in the tables that follow.

The animals required to begin a given experiment were pooled and implanted monolaterally on day 0. Treatments were administered on measurable tumors. The solid tumors were allowed to grow to the desired volume range (animals with tumors not in the desired range were excluded). The mice were then pooled and unselectively distributed to the various treatment and control groups. Treatment started 33 days post CR-LRB-013P tumor fragment implantation as indicated in the results section and in each table. The dosages are expressed in mg/kg, based on the body weight at start of therapy. Mice were checked daily, and adverse clinical reactions noted. Each group of mice was weighed as a whole daily until the weight nadir was reached. Then, groups were weighed once to thrice weekly until the end of the experiment. Tumors were measured with a calliper 2 to 3 times weekly until final sacrifice for sampling time, tumor reached 2000 mm³ or until the animal died (whichever comes first). Solid tumor volumes were estimated from two-dimensional tumor measurements and calculated according to the following equation:

Tumor weight(mg)=Length(mm)×Width²(mm²)/2

The day of death was recorded. Surviving animals were sacrificed and macroscopic examination of the thoracic and abdominal cavities was performed.

A dosage producing a 15% body weight loss (BWL) during three consecutive days (mean of group), 20% BWL during 1 day or 10% or more drug deaths was considered an excessively toxic dosage. Animal body weights included the tumor weight.

The primary efficacy end points are ΔT/ΔC, percent median regression, partial and complete regressions (PR and CR). Statistical analyses were performed on SAS system release 8.2 for SUN4 via Everstat V5 software and SAS 9.2 software. A probability less than 5% (p<0.05) was considered as significant.

### Results of In Vivo Studies

The median tumor burden at start of therapy was 144 to 162 mm³. As single agents, Compound (1) (20 mg/kg/administration (Adm)), Compound (2b) (20 mg/kg/adm) and Compound (2a) (75 mg/kg/adm) were administered PO daily from days 33 to 50 post tumor implantation. In the combination groups, the dose of Compound (1) was combined with each dose of Compound (2a) and Compound (2b), as shown in Table 18.

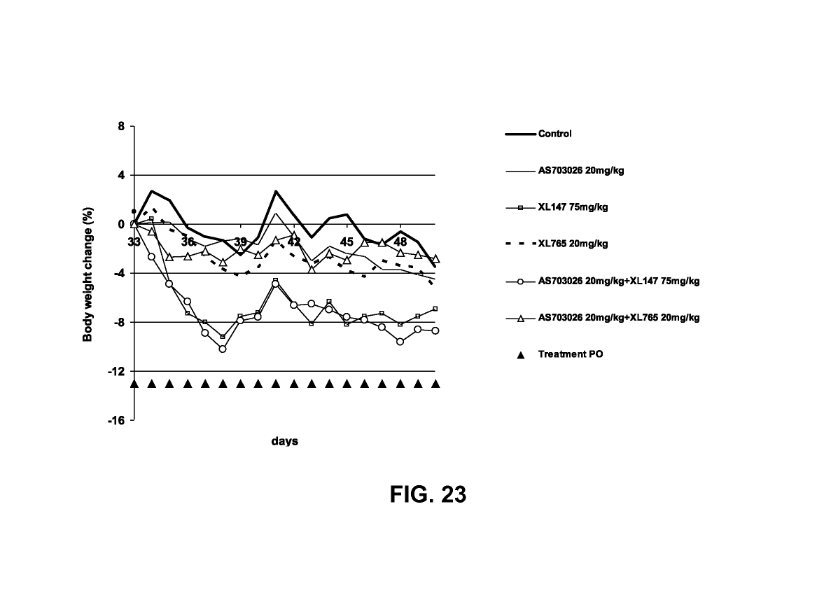

As single agents or used in combination, Compound (1), Compound (2b) and Compound (2a) were tolerated, inducing some BWL but not reaching toxicity (FIG. 23 and Table 18). As single agents under these test conditions, Compound (2a) and Compound (2b) achieved a ΔT/ΔC>40%, while Compound (1) achieved a ΔT/ΔC of 30%.

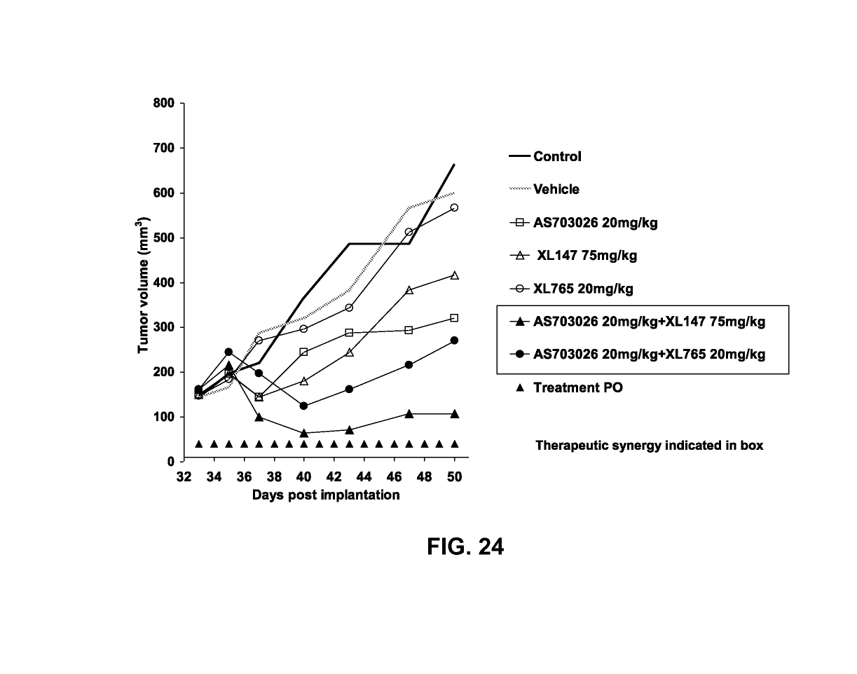

In combination, treatment with Compound (1) at 20 mg/kg/adm and Compound (2b) at 20 mg/kg/adm achieved a ΔT/ΔC of 26% (FIG. 24 and Table 18) with 1/7 partial regression, and as shown by Table 19, therapeutic synergy was reached (p=0.0302 for global analysis). The treatment with Compound (1) at 20 mg/kg/adm and Compound (2a) at 75 mg/kg/adm achieved a ΔT/ΔC of −5% (FIG. 24 and Table 18) with 5/7 partial regression, and as shown by Table 19, therapeutic synergy was achieved (p<0.0001 globally). See also Table 20.

### Summary of In Vivo Results

The in vivo work presented here reports the in vivo antitumor activity of combining Compound (1), an oral potent and selective allosteric inhibitor of MEK1/2, with oral, potent, and specific inhibitors of class I PI3K lipid kinases Compound (2a), a pan-PI3K inhibitor, and Compound (2b), a dual pan-PI3K and mTOR inhibitor. This work has been performed against human primary colon carcinoma CR-LRB-013P xenografts harboring a KRAS mutation.

In the study, combination treatment induced a sustained tumor stasis or partial regressions during the treatment phase and reached therapeutic synergy.

Accordingly, a potent antitumor activity with therapeutic synergy has been achieved in KRAS mutant CR-LRB-013P driven xenograft model when combining the inhibitor of MEK1/2 Compound (1) with Compound (2a), a pan-PI3K inhibitor or Compound (2b), a dual pan-PI3K and mTOR inhibitor.

### Example 7

### Evaluation of Tumor Permeability

The following experiment was conducted to evaluate the impact of Compound (2a) and Compound (2b), alone or in combination with Compound (1), on tumor vascular permeability.

### Methods

HCT116 tumor cells were implanted subcutaneously in the intra-scapular region in SCID mice. Implanted animals received Compound (2a) 50 mg/kg or Compound (2b) 20 mg/kg from day 11 to day 13, as single agents or combined with Compound (1) 10 mg/kg (five animals per group). Each agent was given by oral route on a daily schedule. Tumor growth was monitored throughout the experiment by callipering the tumors. To quantify tumor vascular permeability, tumors were excised under ketamine/Xylazine (120/6 mg/kg ip) anesthesia at day 13, 4 hours post last treatment, 30 min after 0.5% Evans Blue iv injection, and 2 min post Dextran-Fitc 100 mg/kg iv injection. Tumors were then snap frozen, and 25 μm sections obtained for fluorescence quantification. Tumors sections were imaged with Icyte at 488 nm for vascular Dextran-Fitc determination and at 633 nm for Evans-Blue extravasation determination. Respective fluorescence were quantified as the sum of integral phantoms of fluorescence intensity and expressed as the mean ratio of Evans-Blue signal/Dextran-Fitc Signal.

### Results

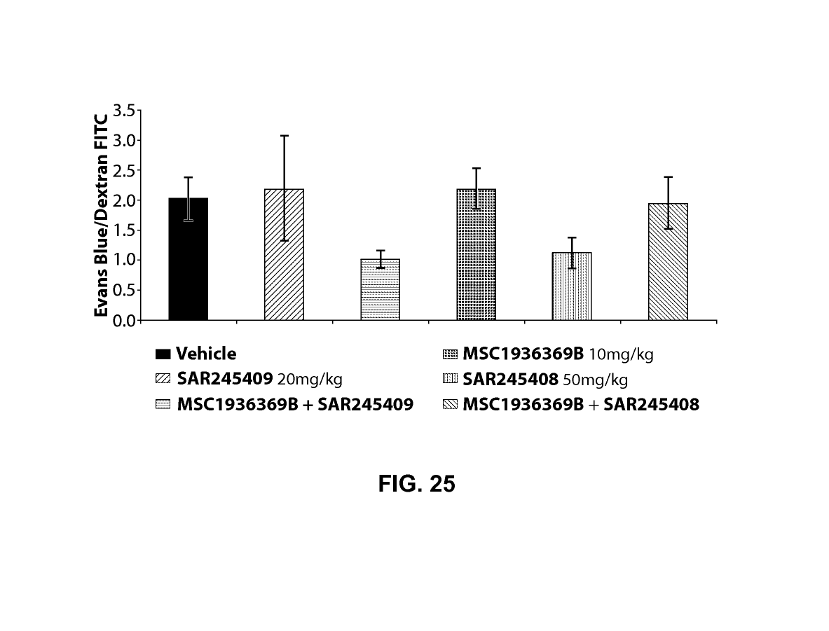

Under these test conditions in advanced subcutaneously grafted HCT116 human KRAS/PI3KCA mutated colon carcinoma, Compound (1) and Compound (2a) used as single agents and the combination of Compound (2a)/Compound (1) did not significantly modify tumor permeability, showing −9%, −8% and 4% decrease, respectively, of the Evans-Blue/Dextran-Fitc ratio compared to control. On the other hand, 3 days of treatment with Compound (2b) or the combination of Compound (2b)/Compound (1) induced clear modulation of Evans-Blue/Dextran Fitc ratio, producing a 50% decrease for the single agent and 45% decrease for the combination. See FIG. 25.

### Summary

Compound (2b) used as a single agent or in combination with Compound (1) alters tumor vascular permeability after 3 days of treatment in advanced subcutaneously grafted HCT116 human KRAS/PI3KCA mutated colon carcinoma. This alteration in HCT116 tumor vascular permeability disrupts in vivo fluorescent-Annexin tumor distribution for FMT imaging and precludes apoptosis detection by this method.

TABLE 1
Results of Compound (1) and Compound (2b) in vitro
combination separated into 5 different groups

| | | | | | |
|---|---|---|---|---|---|
| Cell line | Vector sum | Average | HSA Cumulative | z-score MSC6369 | z-score MSC0765 |

Group I. Cell lines resistant to both MSC6389 and MS00765 (z-score > 1.0)

| | | | | | |
|---|---|---|---|---|---|
| A673 | −3.63 | −0.18 | −0.1% | 3.00 | 0.96 |
| PANC1 | −0.89 | −0.11 | −0.15 | 3.00 | 1.16 |

Group II. Cell lines with relative resistance to both agents (1.0 < z-score < 0)

| | | | | | |
|---|---|---|---|---|---|
| BT20 | −1.10 | −0.14 | −0.23 | 0.02 | 0.32 |
| DLD1 | −0.81 | −0.12 | −0.23 | 0.19 | 0.81 |
| DU145 | −0.81 | −0.11 | −0.17 | 0.73 | −0.05 |

Group III. Cell lines vary resistant to one of the agents (one z-score > 0)

| | | | | | |
|---|---|---|---|---|---|
| CASKI | −0.89 | −0.12 | −0.19 | 1.55 | −0.23 |
| EJ28 | −1.00 | −0.18 | −0.22 | −0.68 | 1.25 |
| HCT116 | −1.19 | −0.13 | −0.19 | −0.32 | 1.90 |

Group IV. Cell lines relatively active for both agents

| | | | | | |
|---|---|---|---|---|---|
| A549 | −0.96 | −0.11 | −0.17 | −0.11 | −0.20 |
| MCF7 | −0.87 | −0.12 | −0.22 | −0.33 | 0.30 |

Group V. Cell lines: very sensitive to one of the agents

| | | | | | |
|---|---|---|---|---|---|
| SKBR3 | −0.85 | −0.07 | −0.08 | 0.69 | −2.18 |

TABLE 2
Antitumor activity of Compound (1) (5 mg/kg) in combination with Compound (2b) (30 mg/kg)
or Compound (2a) (50 and 75 mg/kg) against human HCT 116 bearing SCID female mice

| | | | | | Average body | | | |
|---|---|---|---|---|---|---|---|---|
| | | Dosage in | | Drug | weight change | Median | | |
| | Route/Dosage | mg/kg per | | death | in % per mouse | ΔT/ΔC | | |
| | in mL/kg per | administration | Schedule | (Day of | at nadir (day | in % | Regressions | |
| Agent (batch | administration | (total dose) | in days | death | of nadir | day 18 | Partial | Complete |
| Compound (1) | PO | 5 (40) | 11-18 | 0/7 | −3.4 (14) | 70 | 0/7 | 0/7 |
| (VAC.HAL1.166) | 10 mL/kg | | | | | | | |
| Compound (2b) | PO | 30 (240) | 11-18 | 0/7 | −6.7 (18) | 77 | 1/7 | 0/7 |
| (T1007388) | 10 mL/kg | | | | | | | |
| Compound (2a) | PO | 75 (600) | 11-18 | 0/7 | −8.3 (18) | 80 | 0/7 | 0/7 |
| (20090150) | 10 mL/kg | 50 (400) | | 0/7 | −5.8 (18) | 62 | 0/7 | 0/7 |
| Compound (1) | PO | 5 (40) | 11-18 | 0/7 | −7.4 (15) | 27 | 0/7 | 0/7 |
| Compound (2b) | 10 mL/kg | 30 (240) | | | | | | |
| Compound (1) | PO | 5 (40) | 11-18 | 0/7 | −7.4 (18) | 21 | 0/7 | 0/7 |
| Compound (2a) | 10 mL/kg | 75 (600) | | | | | | |
| | | 5 (40) | 11-18 | 0/7 | −7.4 (16) | 22 | 0/7 | 0/7 |
| | | 50 (400) | | | | | | |
| Control | | | | 0/7 | −0.8 (18) | 100 | 0/7 | 0/7 |

Tumor size at start of therapy was 162-352 mm³, with a median tumor burden per group of 198-221 mm³. Drug formulation: Compound (1) = carboxymethylcellulose 0.5%, tween 20 0.25% in water; Compound (2b) = water, pH 3, Compound (2a) = water. Treatment duration: Compound (1), Compound (2b), Compound (2a) and combination = 8 days. Abbreviations used: BWL = body weight loss, ΔT/ΔC = Ratio of change in tumor volume from baseline median between treated and control groups (TVday − TV0)/(CVday − CV0) * 100, HNTD = highest non toxic dose, HDT = highest dose tested.

TABLE 3
Antitumor activity of Compound (1) (5 mg/kg) in combination with Compound (2b) (30 mg/kg) or Compound (2a)
(50 and 75 mg/kg) against human HCT 116 bearing SCID female mice: Therapeutic synergy determination

| | | Estimated Difference | | |
|---|---|---|---|---|
| Group comparison | Day | Between Groups Means | T-test value | p^{a} |
| Compound (1) 5 mg/kg and Compound (2b) 30 mg/kg | Global | −72.6786 | −1.91 | 0.0606 |
| versus | D 11 | 1.7143 | 0.05 | 0.9572 |
| Compound (2b) at 30 mg/kg | D 14 | −91.4286 | −1.31 | 0.2035 |
| | D 16 | −79.2857 | −1.03 | 0.3134 |
| | D 18 | −121.71 | −1.14 | 0.2653 |
| Compound (1) at 5 mg/kg and Compound (2a) at 50 mg/kg | Global | −93.5357 | −2.66 | 0.0091 |
| versus | D 11 | −4.5714 | −0.14 | 0.8866 |
| Compound (2a) at 50 mg/kg | D 14 | −62.4286 | −0.97 | 0.3394 |
| | D 16 | −128.29 | −1.77 | 0.0853 |
| | D 18 | −178.86 | −1.84 | 0.0735 |
| Compound (1) at 5 mg/kg and Compound (2a) at 75 mg/kg | Global | −156.68 | −4.45 | <.0001 |
| versus | D 11 | −4.1429 | −0.13 | 0.8972 |
| Compound (2a) at 75 mg/kg | D 14 | −32.2857 | −0.50 | 0.6196 |
| | D 16 | −249.14 | −3.44 | 0.0015 |
| | D 18 | −341.14 | −3.52 | 0.0012 |

^{a}Each combination was compared to the best single agent using estimates obtained from a 2-way analysis of variance with repeated measurements (Time factor) on parameter tumor volume (proc mixed of SAS 9.2 software). A probability less than 5% (p < 0.05) was considered as significant.

TABLE 4
Antitumor activity of Compound (1) (10 and 20 mg/kg) in combination with Compound (2b) (20
mg/kg) or Compound (2a) (50 and 75 mg/kg) against human HCT 116 bearing SCID female mice

| | | | | | Average body | | | | |
|---|---|---|---|---|---|---|---|---|---|
| | | Dosage in | | Drug | weight change | Median | Median % of | | |
| | Route/Dosage | mg/kg per | | death | in % per mouse | ΔT/ΔC | regression | | |
| | in mL/kg per | administration | Schedule | (Day of | at nadir (day | in % | on | Regressions | |
| Agent (batch) | administration | (total dose) | in days | death) | of nadir) | day 18 | day 18 | Partial | Complete |
| Compound (1) | PO | 20 (160) | 11-18 | 0/7 | −1.1 (15) | 22 | — | 0/7 | 0/7 |
| (VAC.HAL1.166) | 10 mL/kg | 10 (90) a | | 0/7 | −2.2 (18) | 20 | — | 0/7 | 0/7 |
| Compound (2b) | PO | 20 (160) | | 0/7 | −3.7 (15) | 71 | — | 0/7 | 0/7 |
| (T1007388) | 10 mL/kg | | | | | | | | |
| Compound (2a) | PO | 75 (600) | 11-18 | 0/7 | −6.7 (18) | 56 | — | 0/7 | 0/7 |
| (20090150) | 10 mL/kg | 50 (400) | | 0/7 | −7.7 (18) | 52 | — | 0/7 | 0/7 |
| Compound (1) | PO | 20 (160) | | 0/7 | −4.3 (14) | 0 | — | 0/7 | 0/7 |
| Compound (2b) | 10 mL/kg | 20 (160) | | | | | | | |
| Compound (1) | PO | 20 (160) | | 0/7 | −5.3 (18) | −2 | 8 | 1/7 | 0/7 |
| Compound (2a) | 10 mL/kg | 50 (400) | | | | | | | |
| Compound (1) | PO | 10 (80) | | 0/7 | −6.3 (18) | 0 | — | 2/7 | 0/7 |
| Compound (2b) | 10 mL/kg | 20 (160) | | | | | | | |
| Compound (1) | PO | 10 (80) | 11-18 | 0/7 | −6.3 (18) | 5 | — | 1/7 | 0/7 |
| Compound (2a) | 10 mL/kg | 75 (600) | | | | | | | |
| | | 10 (80) | | 0/7 | −8.0 (18) | −4 | 8 | 1/7 | 0/7 |
| | | 50 (400) | | | | | | | |
| Control | | | | 0/7 | −2.4 (13) | 100 | | | |

Tumor size at start of therapy was 126-294 mm³, with a median tumor burden per group of 180-198 mm³. Drug formulation: Compound (1) = carboxymethylcellulose 0.5%, Tween 20 0.25% in water; Compound (2b) = water, pH 3; Compound (2a) = water. Treatment duration: Compound (1), Compound (2b), Compound (2a) and combination = 8 days. Abbreviations used: BWL = body weight loss, ΔT/ΔC = Ratio of change in tumor volume from baseline median between treated and control groups (TVday − TV0)/(CVday − CV0) * 100, HNTD = highest non toxic dose, HDT = highest dose tested.
a On day 17, mice received 20 mg/kg instead of 10 mg/kg.

TABLE 5
Antitumor activity of Compound (1) (10 and 20 mg/kg) in combination with
Compound (2b) (20 mg/kg) or Compound (2a) (50 and 75 mg/kg) against human
HCT 116 bearing SCID female mice: therapeutic synergy determination

| | | Estimated Difference | | |
|---|---|---|---|---|
| Group comparison | Day | Between Groups Means | T-test value | p^{a} |
| Compound (1) at 10 mg/kg and Compound | Global | −107.10 | −3.71 | 0.0004 |
| (2b) at 20 mg/kg | D 11 | 0.5714 | 0.02 | 0.9828 |
| versus | D 14 | −160.14 | −2.91 | 0.0061 |
| Compound (1) at 10 mg/kg | D 18 | −161.71 | −2.62 | 0.0128 |
| Compound (1) at 20 mg/kg and Compound | Global | −35.9524 | −1.24 | 0.2169 |
| (2b) at 20 mg/kg | D 11 | 8.2857 | 0.32 | 0.7545 |
| versus | D 14 | −50.4286 | −0.92 | 0.3648 |
| Compound (1) at 20 mg/kg | D 18 | −65.7143 | −1.07 | 0.2940 |
| Compound (1) at 10 mg/kg and Compound | Global | −106.10 | −3.21 | 0.0019 |
| (2a) at 50 mg/kg | D 11 | 4.1429 | 0.15 | 0.8784 |
| versus | D 14 | −139.29 | −2.90 | 0.0056 |
| Compound (1) at 10 mg/kg | D 18 | −183.14 | −2.22 | 0.0315 |
| Compound (1) at 10 mg/kg and Compound | Global | −92.5238 | −2.80 | 0.0063 |
| (2a) at 75 mg/kg | D 11 | −5.1429 | −0.19 | 0.8494 |
| versus | D 14 | −158.86 | −3.31 | 0.0018 |
| Compound (1) at 10 mg/kg | D 18 | −113.57 | −1.37 | 0.1758 |

^{a}Each combination was compared to the best single agent using estimates obtained from a two-way analysis of variance with repeated measurements (Time factor) on parameter tumor volume (proc mixed of SAS 9.2 software). A probability less than 5% (p < 0.05) was considered as significant.

TABLE 6
Antitumor activity of Compound (1) (10 and 20 mg/kg) in combination with Compound
(2a) (50 and 75 mg/kg) against human HCT 116 bearing SCID female mice

| | | | | | Average body | | | | |
|---|---|---|---|---|---|---|---|---|---|
| | | Dosage in | | Drug | weight change | Median | Median % of | | |
| | Route/Dosage | mg/kg per | | death | in % per mouse | ΔT/ΔC | regression | | |
| | in mL/kg per | injection | Schedule | (Day of | at nadir (day | in % | on | Regressions | |
| Agent (batch) | injection | (total dose) | in days | death) | of nadir) | day 20 | day 20 | Partial | Complete |
| Compound (1) | PO | 20 (200) | 11-20 | 0/10 | −3.6 (19) | 34 | — | 0/10 | 0/10 |
| (VAC.HAL1.166) | 10 mL/kg | | | | | | | | |
| (27) | | 10 (100) | | 0/10 | −4.9 (20) | 43 | — | 0/10 | 0/10 |
| Compound (2a) | PO | 75 (750) | 11-20 | 0/10 | −8.5 (20) | 64 | — | 0/10 | 0/10 |
| (20090150) | 10 mL/kg | 50 (500) | | 0/10 | −7.8 (19) | 66 | — | 0/10 | 0/10 |
| Compound (1) | PO | 20 (380) | 11-29b | 0/10 | −7.8 (17) | 9 | | 0/10 | 0/10 |
| Compound (2a) | 10 mL/kg | 75 (1425) | | | | | — | | |
| | | 20 (200) | 11-20 | 0/10 | −5.6 (20) | 22 | | 0/10 | 0/10 |
| | | 50 (500) | | | | | — | | |
| Compound (1) | PO | 10 (100) | 11-20 | 0/10 | −7.5 (20) | 18 | | 0/10 | 0/10 |
| Compound (2a) | 10 mL/kg | 75 (750) | | | | | — | | |
| | | 10 (100) | 11-20 | 0/10 | −7.3 (20) | 19 | | 0/10 | 0/10 |
| | | 50 (500) | | | | | — | | |
| Control | | | — | 0/10 | −1.4 (20) | — | | | |
| Vehicle | | | 11-20 | 0/10 | −3.1 (20) | — | | | |

Tumor size at start of therapy was 112-319 mm3, with a median tumor burden per group of 187-189 mm3. Drug formulation: Compound (1) = carboxymethylcellulose 0.5%, Tween 20 0.25% in water; Compound (2a) = water. Treatment duration: Compound (1), Compound (2a) and combination = 10 days. Abbreviations used: bwl = body weight loss, ΔT/ΔC = (TVday − TV0)/(CVday − CV0) * 100, HNTD = highest non toxic dose, HDT = highest dose tested.

TABLE 7
Antitumor activity of Compound (1) (10 and 20 mg/kg) in combination
with Compound (2a) (50 and 75 mg/kg) against human HCT 116 bearing
SCID female mice: therapeutic synergy determination

| | | Estimated Difference | | |
|---|---|---|---|---|
| Group comparison | Day | Between Groups Means | T-test value | p^{a} |
| Combination of Compound (1) at 20 | Global | −186.00 | −3.80 | 0.0003 |
| mg/kg and Compound (2a) at 75 mg/kg | D 14 | −117.00 | −2.60 | 0.0110 |
| versus Compound (1) at 20 mg/kg alone | D 18 | −183.70 | −3.09 | 0.0027 |
| | D 20 | −257.30 | −3.02 | 0.0034 |
| Combination of Compound (1) at 20 | Global | −87.7667 | −1.79 | 0.0764 |
| mg/kg and Compound (2a) at 50 mg/kg | D 14 | −95.3000 | −2.12 | 0.0372 |
| versus Compound (1) at 20 mg/kg alone | D 18 | −73.1000 | −1.23 | 0.2219 |
| | D 20 | −94.9000 | −1.11 | 0.2692 |
| Combination of Compound (1) at 10 | Global | −127.30 | −2.60 | 0.0109 |
| mg/kg and Compound (2a) at 75 mg/kg | D 14 | −68.9000 | −1.53 | 0.1297 |
| versus Compound (1) at 10 mg/kg alone | D 18 | −99.6000 | −1.68 | 0.0974 |
| | D 20 | −213.40 | −2.50 | 0.0143 |
| Combination of Compound (1) at 10 | Global | −131.30 | −2.68 | 0.0088 |
| mg/kg and Compound (2a) at 50 mg/kg | D 14 | −104.60 | −2.32 | 0.0226 |
| versus Compound (1) at 10 mg/kg | D 18 | −140.30 | −2.36 | 0.0206 |
| | D 20 | −149.00 | −1.75 | 0.0844 |

^{a}Each combination was compared to the best single agent using estimates obtained from a 2-way analysis of variance with repeated measurements (Time factor) on parameter tumor volume (proc mixed of SAS 9.2 software). A probability less than 5% (p < 0.05) was considered as significant.

TABLE 8
Antitumor activity of Compound (1) (10 and 20 mg/kg) in combination with
Compound (2b) (20 mg/kg) against human HCT 116 bearing SCID female mice

| | | | | | Average body | | | | |
|---|---|---|---|---|---|---|---|---|---|
| | | Dosage in | | Drug | weight change | Median | Median % of | | |
| | Route/Dosage | mg/kg per | | death | in % per mouse | ΔT/ΔC | regression | | |
| | in mL/kg per | injection | Schedule | (Day of | at nadir (day | in % | on | Regressions | |
| Agent (batch) | injection | (total dose) | in days | death) | of nadir) | day 20 | day 20 | Partial | Complete |
| Compound (1) | PO | 20 (200) | 11-20 | 0/10 | −4.1 (18) | 41 | — | 0/10 | 0/10 |
| (VAC. HAL1.166) | 10 mL/kg | | | | | | | | |
| (27) | | 10 (100) | | 0/10 | −2.3 (13) | 53 | — | 0/10 | 0/10 |
| Compound (2b) | PO | 20 (200) | 11-20 | 0/10 | −4.8 (20) | 83 | — | 0/10 | 0/10 |
| (T1007388) | 10 mL/kg | | | | | | | | |
| Compound (1) | PO | 20 (200) | 11-20 | 0/10 | −3.3 (13) | 15 | — | 0/10 | 0/10 |
| Compound (2b) | 10 mL/kg | 20 (200) | | | | | | | |
| | | 10 (100) | | 0/10 | −3.7 (13) | 30 | — | 0/10 | 0/10 |
| | | 20 (200) | | | | | | | |
| Control | | | | 0/10 | −4.3 (20) | — | | | |
| Vehicle | | | 11-20 | 0/10 | −2.1 (16) | — | | | |

Tumor size at start of therapy was 144-294 mm³, with a median tumor burden per group of 189-196 mm3. Drug formulation: Compound (1) = carboxymethylcellulose 0.5%, Tween 20 0.25% in water; Compound (2b) = water, pH 3. Treatment duration: Compound (1), Compound (2b) and combination = 10 days. Abbreviations used: bwl = body weight loss, ΔT/ΔC = (TVday − TV0)/(CVday − CV0) * 100, HNTD = highest non toxic dose, HDT = highest dose tested

TABLE 9
Antitumor activity of Compound (1) (10 and 20 mg/kg) in combination
with Compound (2b) (20 mg/kg) against human HCT 116 bearing
SCID female mice: therapeutic synergy determination

| | | Estimated Difference | T-test | |
|---|---|---|---|---|
| Group comparison | Day | Between Groups Means | value | p^{a} |
| Combination of Compound (1) at | Global | −180.40 | −3.53 | 0.0008 |
| 20 mg/kg and Compound (2b) at | D 13 | −95.2000 | −2.25 | 0.0281 |
| 20 mg/kg versus Compound (1) at | D 15 | −168.90 | −3.09 | 0.0032 |
| 20 mg/kg alone | D 18 | −194.70 | −2.45 | 0.0172 |
| | D 20 | −262.80 | −2.78 | 0.0072 |
| Combination of Compound (1) at | Global | −202.72 | −3.97 | 0.0002 |
| 10 mg/kg and Compound (2b) at | D 13 | −51.3000 | −1.22 | 0.2295 |
| 20 mg/kg versus Compound (1) at | D 15 | −212.90 | −3.89 | 0.0003 |
| 10 mg/kg alone | D 18 | −272.10 | −3.43 | 0.0011 |
| | D 20 | −274.60 | −2.91 | 0.0051 |

^{a}Each combination was compared to the best single agent using estimates obtained from a 2-way analysis of variance with repeated measurements (Time factor) on parameter tumor volume (proc mixed of SAS 9.2 software). A probability less than 5% (p < 0.05) was considered as significant.

TABLE 10
Percent ΔT/ΔC and statistical analysis in MiaPaCa-2 tumor-bearing mice
treated with Compound (1), Compound (2a), and Compound (2b) alone or in combination.

| | | | | | | AS703026 + | AS703026 + |
|---|---|---|---|---|---|---|---|
| | % ΔT/ΔC | Vehicle | AS703026 | XL-147 | XL-765 | XL-147 | XL-765 |
| | | po QD | 5 mg/kg po QD | 50 mg/kg po QD | 30 mg/kg po QD | po QD | po QD |
| Vehicle po QD | 100.0 | | <0.05 | <0.05 | <0.05 | <0.05 | <0.05 |
| AS703026 5 mg/kg po QD | 56.2 | <0.05 | | NS | NS | NS | <0.05 |
| XL-147 50 mg/kg po QD | 71.2 | <0.05 | NS | | NS | NS | <0.05 |
| XL-765 30 mg/kg po QD | 77.0 | <0.05 | NS | NS | | NS | <0.05 |
| AS703026 + XL-147 | 48.7 | <0.05 | NS | NS | NS | | NS |
| AS703026 + XL-765 | 27.3 | <0.05 | <0.05 | <0.05 | <0.05 | NS | |

The mean percent of actual Miapaca-2 tumor growth inhibited by the treatments was calculated as follows: [% ΔT/ΔC = (TV_{f} − TVᵢ/TV_{fCtrl} − TV_{iCtrl}) × 100%], where TV = tumor volume, f = final, i = initial and Ctrl = control group.

TABLE 11A

| | ΔT/ΔC (%) |
|---|---|
| | on d 18 |
| Compound (1) | 70 |
| 5 mpk | |
| Compound (2b) | 77 |
| 30 mpk | |
| Compound (2b) | 27 |
| 30 mpk | |
| Compound (1) | |
| 5 mpk | |

TABLE 11B

| | ΔT/ΔC (%) |
|---|---|
| | on d 18 |
| Compound (1) | 70 |
| 5 mpk | |
| Compound (2a) | 80 |
| 75 mpk | |
| Compound (2a) | 62 |
| 50 mpk | |
| Compound (2a) | 21 |
| 75 mpk | |
| Compound (1) | |
| 5 mpk | |
| Compound (2a) | 22 |
| 50 mpk | |
| Compound (1) | |
| 5 mpk | |

TABLE 12

| | ΔT/ΔC (%) |
|---|---|
| | on d 18 |
| Compound (1) | 22 |
| 20 mpk | |
| Compound (1) | 20 |
| 10 mpk | |
| Compound (2b) | 71 |
| 20 mpk | |
| Compound (2b) | 0 |
| 20 mpk | |
| Compound (1) | |
| 20 mpk | |
| Compound (2b) | 0 |
| 20 mpk | |
| Compound (1) | |
| 10 mpk | |

TABLE 12B

| | ΔT/ΔC (%) |
|---|---|
| | on d 18 |
| Compound (1) | 20 |
| 10 mpk | |
| Compound (2a) | 56 |
| 75 mpk | |
| Compound (2a) | 52 |
| 50 mpk | |
| Compound (2a) | 5 |
| 75 mpk | |
| Compound (1) | |
| 10 mpk | |
| Compound (2a) | −4 |
| 50 mpk | |
| Compound (1) | |
| 10 mpk | |

TABLE 13

| | ΔT/ΔC (%) |
|---|---|
| | on d 20 |
| Compound (1) | 34 |
| 20 mpk | |
| Compound (1) | 43 |
| 10 mpk | |
| Compound (2a) | 64 |
| 75 mpk | |
| Compound (2a) | 66 |
| 50 mpk | |
| Compound (2a) | 9 |
| 75 mpk | |
| Compound (1) | |
| 20 mpk | |
| Compound (2a) | 18 |
| 75 mpk | |
| Compound (1) | |
| 10 mpk | |
| Compound (2a) | 22 |
| 50 mpk | |
| Compound (1) | |
| 20 mpk | |
| Compound (2a) | 19 |
| 50 mpk | |
| Compound (1) | |
| 10 mpk | |

TABLE 14

| | ΔT/ΔC (%) |
|---|---|
| | on d 20 |
| Compound (1) | 41 |
| 20 mpk | |
| Compound (1) | 53 |
| 10 mpk | |
| Compound (2b) | 83 |
| 20 mpk | |
| Compound (2b) | 15 |
| 20 mpk | |
| Compound (1) | |
| 20 mpk | |
| Compound (2b) | 30 |
| 20 mpk | |
| Compound (1) | |
| 10 mpk | |

TABLE 15
Antitumor activity of Compound (1) (20 mg/kg) in combination with Compound (2b) (20 mg/kg) or Compound
(2a) (75 mg/kg) against human primary colon CR-LRB-009C tumors bearing SCID female mice

| | | | | | Average body | | | |
|---|---|---|---|---|---|---|---|---|
| | | Dosage in | | Drug | weight change | Median | | |
| | Route/Dosage | mg/kg per | | death | in % per mouse | ΔT/ΔC | | |
| | in mL/kg per | administration | Schedule | (Day of | at nadir (day | in % | Regressions | |
| Agent (batch) | administration | (total dose) | in days | death) | of nadir) | day 21 | Partial | Complete |
| Compound (1) | PO | 20 (220) | 11-21 | 0/7 | −7.7 (20) | 53 | 0/7 | 0/7 |
| (VAC.HAL1.166) | 10 mL/kg | | | | | | | |
| Compound (2b) | PO | 20 (220) | 11-21 | 0/7 | −7.4 (19) | 51 | 0/7 | 0/7 |
| (T1007388) | 10 mL/kg | | | | | | | |
| Compound (2a) | PO | 75 (825) | 11-21 | 0/7 | −15.8 (21) | 39 | 0/7 | 0/7 |
| (T1007032 | 10 mL/kg | | | | | | | |
| M022906) | | | | | | | | |
| Compound (1) | PO | 20 (220) | 11-21 | 0/7 | −13.7 (21) | 4 | 1/7 | 0/7 |
| Compound (2b) | 10 mL/kg | 20 (220) | | | | | | |
| Compound (1) | PO | 20 (220) | 11-21 | 0/7 | −14.0 (21) | 21 | 0/7 | 0/7 |
| Compound (2a) | 10 mL/kg | 75 (825) | | | | | | |
| Control | | | | 0/7 | −7.8 (20) | 100 | | |

Tumor size at start of therapy was 100-221 mm³, with a median tumor burden per group of 126-144 mm³. Drug formulation: Compound (1) = carboxymethylcellulose 0.5%, tween 20 0.25% in water; Compound (2b) and Compound (2a) = water, pH 3. Treatment duration: Compound (1), Compound (2a) and Compound (2b) and combination = 11 days. Abbreviations used: BWL = body weight loss, ΔT/ΔC = Ratio of change in tumor volume from baseline median between treated and control groups (TVday − TV0)/(CVday − CV0) * 100, HDT = highest dose tested.

TABLE 16
Antitumor activity of Compound (1) (20 mg/kg) in combination with Compound
(2b) (20 mg/kg) or Compound (2a) (75 mg/kg) against human primary colon CR-
LRB-009C tumors bearing SCID female mice: Therapeutic synergy determination

| | | Estimated Difference | | |
|---|---|---|---|---|
| | Day | between Groups Means | T-test value | p^{a} |
| Combination of Compound (1) at 20 mg/kg and Compound (2a) | Global | −21.3214 | −2.13 | 0.0386 |
| at 75 mg/kg | D 13 | 3.5714 | 0.27 | 0.7891 |
| versus | D 15 | −22.7143 | −1.71 | 0.0912 |
| Compound (2a) at 75 mg/kg | D 18 | −34.5714 | −2.60 | 0.0109 |
| | D 21 | −31.5714 | −2.37 | 0.0197 |
| Combination of Compound (1) at 20 mg/kg and Compound | Global | −56.3016 | −5.61 | <.0001 |
| (2b) at 20 mg/kg | D 13 | −5.1429 | −0.39 | 0.7001 |
| versus | D 15 | −57.2857 | −4.30 | <.0001 |
| Compound (2b) at 20 mg/kg | D 18 | −82.2143 | −6.18 | <.0001 |
| | D 21 | −80.5635 | −5.89 | <.0001 |

^{a}Each combination was compared to the best single agent using estimates obtained from a 2-way analysis of variance with repeated measurements (Time factor) on parameter tumor volume (proc mixed of SAS 9.2 software). A probability less than 5% (p < 0.05) was considered as significant.

TABLE 17

| | ΔT/ΔC (%) |
|---|---|
| | on d 21 |
| Compound (1) 20 mg/kg | 53 |
| Compound (2a) 75 mg/kg | 39 |
| Compound (2b) 20 mg/kg | 51 |
| Compound (2a) 75 mg/kg | 21 |
| Compound (1) 20 mg/kg | |
| Compound (2b) 20 mg/kg | 4 |
| Compound (1) 20 mg/kg | |

TABLE 18
Antitumor activity of Compound (1) (20 mg/kg) in combination with Compound (2b) (20 mg/kg) or Compound
(2a) (75 mg/kg) against human primary colon CR-LRB-013P tumors bearing SCID female mice

| | | | | | Average body | | | |
|---|---|---|---|---|---|---|---|---|
| | | Dosage in | | Drug | weight change | Median | | |
| | Route/Dosage | mg/kg per | | death | in % per mouse | ΔT/Δ C | | |
| | in mL/kg per | administration | Schedule | (Day of | at nadir (day | in % | Regressions | |
| Agent (batch) | administration | (total dose) | in days | death) | of nadir) | day 50 | Partial | Complete |
| Compound (1) | PO | 20 (360) | 33-50 | 0/7 | −4.5 (50) | 30 | 0/7 | 0/7 |
| (VAC.HAL1.166) | 10 mL/kg | | | | | | | |
| Compound (2b) | PO | 20 (360) | 33-50 | 0/7 | −5.2 (50) | 83 | 0/7 | 0/7 |
| (T1007388) | 10 mL/kg | | | | | | | |
| Compound (2a) | PO | 75 (1350) | 33-50 | 0/7 | −9.2 (50) | 53 | 0/7 | 0/7 |
| (20090150) | 10 mL/kg | | | | | | | |
| Compound (1) | PO | 20 (360) | 33-50 | 0/7 | −3.7 (43) | 26 | 1/7 | 0/7 |
| Compound (2b) | 10 mL/kg | 20 (360) | | | | | | |
| Compound (1) | PO | 20 (360) | 33-50 | 0/7 | −10.2 (38) | −5 | 5/7 | 0/7 |
| Compound (2a) | 10 mL/kg | 75 (1350) | | | | | | |
| Control | | | | 0/7 | −3.5 (50) | 100 | | |

Tumor size at start of therapy was 108-245 mm³, with a median tumor burden per group of 144-162 mm³. Drug formulation: Compound (1) = carboxymethylcellulose 0.5%, tween 20 0.25% in water; Compound (2b) and Compound (2a) = water, pH 3. Treatment duration: Compound (1), Compound (2a) and Compound (2b) and combination = 18 days. Abbreviations used: BWL = body weight loss, ΔT/ΔC = Ratio of change in tumor volume from baseline median between treated and control groups (TVday − TV0)/(CVday − CV0) * 100, HDT = highest dose tested.

TABLE 19
Antitumor activity of Compound (1) (20 mg/kg) in combination
with Compound (2b) (20 mg/kg) or Compound (2a) (75 mg/kg)
against human primary colon CR-LRB-013P tumors bearing
SCID female mice: Therapeutic synergy determination

| | | Estimated Difference | | |
|---|---|---|---|---|
| | Day | between Groups Means | t Value | p^{a} |
| Combination of | Global | −149.52 | −4.88 | <.0001 |
| Compound (1) at | D 35 | −10.1429 | −0.30 | 0.7639 |
| 20 mg/kg and | D 37 | −67.5714 | −2.18 | 0.0345 |
| Compound (2a) at | D 40 | −191.71 | −6.68 | <.0001 |
| 75 mg/kg versus | D 43 | −199.86 | −4.96 | <.0001 |
| Compound (1) at | D 47 | −207.86 | −3.51 | 0.0011 |
| 20 mg/kg | D 50 | −220.00 | −3.18 | 0.0028 |
| Combination of | Global | −68.5952 | −2.24 | 0.0302 |
| Compound (1) at | D 35 | 8.8571 | 0.26 | 0.7931 |
| 20 mg/kg and | D 37 | 49.0000 | 1.58 | 0.1205 |
| Compound (2b) at | D 40 | −115.71 | −4.03 | 0.0002 |
| 20 mg/kg versus | D 43 | −129.71 | −3.22 | 0.0026 |
| Compound (1) at | D 47 | −122.43 | −2.07 | 0.0450 |
| 20 mg/kg | D 50 | −101.57 | −1.47 | 0.1499 |

^{a}Each combination was compared to the best single agent using estimates obtained from a 2-way analysis of variance with repeated measurements (Time factor) on parameter tumor volume (proc mixed of SAS 9.2 software). A probability less than 5% (p < 0.05) was considered as significant.

TABLE 20

| | ΔT/ΔC (%) |
|---|---|
| | on d 50 |
| Compound (1) 20 mg/kg | 30 |
| Compound (2a) 75 mg/kg | 53 |
| Compound (2b) 20 mg/kg | 83 |
| Compound (2a) 75 mg/kg | −5 |
| Compound (1) 20 mg/kg | |
| Compound (2b) 20 mg/kg | 26 |
| Compound (1) 20 mg/kg | |

While there have been shown and described what are at present considered the preferred embodiments of the invention, those skilled in the art may make various changes and modifications which remain within the scope of the appended claims.

We claim:

1. A composition comprising a compound having the following structural formula: 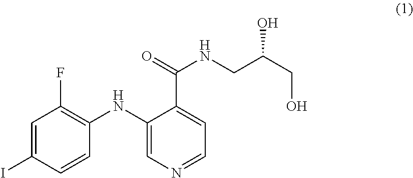 [SMILES: O=C(NC[C@H](O)CO)c1ccncc1Nc1ccc(I)cc1F]
or a pharmaceutically acceptable salt thereof, and a compound having a structural formula selected from the group consisting of 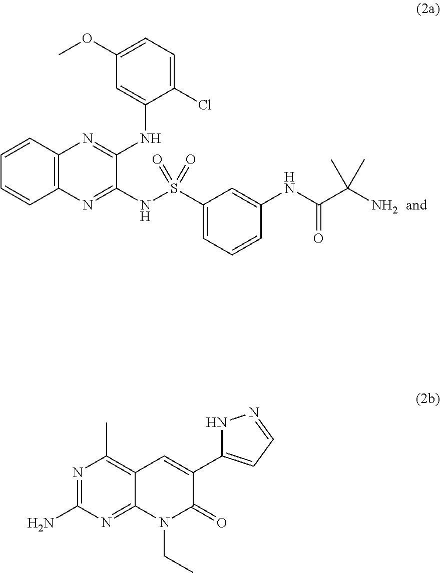 [SMILES: COc1ccc(Cl)c(Nc2nc3ccccc3nc2NS(=O)(=O)c2cccc(NC(=O)C(C)(C)N)c2)c1 ; CCn1c(=O)c(-c2ccn[nH]2)cc2c(C)nc(N)nc21]
or a pharmaceutically acceptable salt thereof.

2. The composition of claim 1, further comprising a pharmaceutically acceptable carrier.

3. The composition of claim 1, wherein said compound according to formula (1) and said compound according to formula (2a) or (2b) are in amounts that produce a synergistic effect in reducing tumor volume in a patient when said composition is administered to a patient.

4. A method of treating a patient with cancer, comprising administering to said patient a therapeutically effective amount of the compound of Formula (1), or a pharmaceutically acceptable salt thereof, in combination with the compound of Formula (2a) or Formula (2b), or a pharmaceutically acceptable salt thereof.

5. The method of claim 4, wherein the effective amount achieves a synergistic effect in reducing a tumor volume in said patient.

6. The method of claim 4, wherein the effective amount achieves tumor stasis in said patient.

7. The method of claim 4, wherein said cancer is selected from the group consisting of non-small cell lung cancer, breast cancer, pancreatic cancer, liver cancer, prostate cancer, bladder cancer, cervical cancer, thyroid cancer, colorectal cancer, liver cancer, muscle cancer, hematological malignancies, melanoma, endometrial cancer and pancreatic cancer.

8. The method of claim 4, wherein the cancer is selected from the group consisting of colorectal cancer, endometrial cancer, hematological malignancies, thryoid cancer, breast cancer, melanoma, pancreatic cancer and prostate cancer.

9. The method of claim 4, wherein said method comprises administering the compound of Formula (2a).

10. The method of claim 4, wherein said method comprises administering the compound of Formula (2b).

11. A kit comprising: (A) the compound of Formula (1), or a pharmaceutically acceptable salt thereof; (B) the compound of Formula (2a) or Formula (2b), or a pharmaceutically acceptable salt thereof; and (C) instructions for use.
